(19) United States
(12) Patent Application Publication
(10) Pub. No.: US 2023/0375538 A1
(43) Pub. Date: Nov. 23, 2023

(54) DUAL BARCODE INDEXES FOR MULTIPLEX SEQUENCING OF ASSAY SAMPLES SCREENED WITH MULTIPLEX INSOLUTION PROTEIN ARRAY

(71) Applicant: ARIZONA BOARD OF REGENTS ON BEHALF OF ARIZONA STATE UNIVERSITY, Scottsdale, AZ (US)

(72) Inventors: Joshua LABAER, Chandler, AZ (US); Jin PARK, Phoenix, AZ (US); Femina RAUF, Tempe, AZ (US)

(21) Appl. No.: 18/017,563

(22) PCT Filed: Jul. 22, 2021
(86) PCT No.: PCT/US2021/042784

§ 371 Date: Jan. 23, 2023

Related U.S. Application Data
(60) Provisional application No. 63/056,282, filed on Jul. 24, 2020.

(51) Int. Cl. G01N 33/537; G01N 33/58

(52) U.S. Cl. CPC G01N 33/537; G01N 33/58; G01N 2458/10

(57) ABSTRACT

Provided herein are compositions comprising coordinated sets of unique DNA barcodes and methods for using the same for multiplex detection and measurement of multiple target molecules in multiple samples using a single next-generation sequencing reaction. In particular, methods are provided in which unique DNA barcodes linked to affinity reagents are contacted to a sample to bind antigens if present in said sample, and then a PCR-based amplification reaction adds barcoded index sequences that contain universal sequencing adaptors as well as unique barcode sequences and amplifies affinity reagent-bound targets for DNA sequencing.

### CROSS-REFERENCE TO RELATED APPLICATIONS

This application claims the benefit of U.S. Appl. No. 63/056,282, filed on Jul. 24, 2020, the content of which is incorporated herein by reference in its entirety.

### STATEMENT REGARDING FEDERALLY SPONSORED RESEARCH OR DEVELOPMENT

This invention was made with government support under R21 CA196442 awarded by the National Institutes of Health. The government has certain rights in the invention.

### BACKGROUND

With the advent of various ‘omics’ technologies and methods which stratify samples and diseases based on measuring many variables simultaneously, there is an increasing demand for high throughput tools that quantify specific targets. There are already numerous genomics tools that assess gene expression, gene copy number, mutations, etc. at a global scale to determine subtypes of disease that might be useful for prognostication and management of therapy. But it is well known that the genome (which is a blue print) does not always reflect the actual state of biology at any time and gene measurements are not always possible from readily accessible samples like blood. Thus, there is a strong desire to have similar high throughput tools to measure the proteome, which is the product of the genome and more closely reflects the current state of biology. However, high throughput measurement of the proteome is much more challenging than similar genome measurements, because there is no protein equivalent to the base pairing measurements that emerge from the inherent double-stranded nature of DNA.

There are a wide variety of methods to measure proteins. These can be generally divided into antibody-based methods and chemistry-based methods. By far, the most common chemistry-based method is mass spectrometry, which is most commonly employed by ionizing peptides (created by proteolytic digestion) and measuring their mobility in a magnetic field. The accuracy of these instruments is sufficient to identify virtually any protein by comparing its spectrum to spectrums predicted from the genome. Although nearly universal in its ability to detect proteins and even modified proteins, mass spectrometry is very low throughput. A thorough examination of a single sample can take hours and it requires great care to run a set samples in a fashion that allows comparison of one run to the next. There are many other tools that detect proteins chemically, but they are not capable of identifying specific proteins in a universal manner.

Detection of proteins is most commonly accomplished with antibodies (or more generally, affinity reagents), and include many different configurations such as western blots, immunoprecipitation, flow cytometry, reverse phase protein arrays, enzyme linked immunosorbent assay (ELISA), and many others. These applications all rely on antibodies that recognize specific targets, and which can bind with extraordinary selectivity and affinity. There are currently more than 2,000,000 antibodies available on the market that target a large fraction of the human proteome. It is important to note that not all antibodies are high quality, but many are quite good and methods to produce antibodies have become routine. Although the use of an antibody to measure its target can be relatively fast, it is not straightforward to multiplex measurements using many antibodies simultaneously. Accordingly, there remains a need in the art for improved, cost-effective methods for simultaneous multiplex detection and measurement of many proteins or other target molecules in multiple samples, including pooled samples.

### BRIEF SUMMARY OF THE DISCLOSURE

In a first aspect, provided herein is a composition comprising, or consisting essentially of, (i) a plurality of modified affinity reagents, each affinity reagent of the plurality comprising a unique identifying nucleotide sequence relative to other affinity reagents of the plurality, wherein each identifying nucleotide sequence is flanked by a first amplifying nucleotide sequence and a second amplifying nucleotide sequence; (ii) a first (e.g., a forward) barcoded index primer comprising a universal sequence A, a first unique index nucleotide sequence, and a sequence configured to anneal to the first amplifying nucleotide sequence; and (iii) a second (e.g., a reverse) barcoded index sequence comprising a universal sequence B, a second unique index nucleotide sequence, and sequence configured to anneal to the second amplifying nucleotide sequence. The first barcoded index primer can be selected from SEQ ID NO:204-SEQ ID NO:233. The second barcoded index primer can be selected from SEQ ID NO:234-SEQ ID NO:253. Identifying nucleotide sequences can be selected from SEQ ID NO:1 and barcode sequences set forth in Table 1. Affinity reagents of the plurality can be antibodies. Affinity reagents of the plurality can be peptide aptamers or nucleic acid aptamers. An identifying nucleotide sequence (e.g., a linker) can be attached to an affinity reagent by a linker comprising a cleavable protein photocrosslinker. An identifying nucleotide sequence can be attached to an affinity reagent by a linker comprising a fluorescent moiety.

In another aspect, provided herein is a method for high throughput multiplex identification and quantification of target molecules in a plurality of samples, comprising or consisting essentially of, (a) for each of a plurality of samples, contacting the sample with a plurality of modified affinity reagents under conditions that promote binding of the modified affinity reagents to target molecules if present in the contacted sample, wherein each modified affinity reagent of the plurality comprises a unique identifying nucleotide sequence relative to other affinity reagents of the plurality, wherein each identifying nucleotide sequence is flanked by a first amplifying nucleotide sequence and a second amplifying nucleotide sequence; (b) contacting the contacted samples of step (a) to a first (e.g., a forward) barcoded index primer and a second (e.g., reverse) barcoded index primer under conditions that promote annealing of the first barcoded index primer and the second barcoded index primer to the first and second amplifying nucleotide sequences, wherein the first barcoded index primer comprises a universal sequence A, a first unique index nucleotide sequence, and a sequence configured to anneal to the first amplifying nucleotide sequence, and wherein the second barcoded index primer comprises a universal sequence B, a second unique index nucleotide sequence, and a sequence configured to anneal to the second amplifying nucleotide sequence; (c) amplifying the contacted samples of (b) to produce an amplified product; and (d) sequencing the amplified product whereby target molecules of each of the plurality of samples is identified and quantified based on detection of the identifying nucleotide sequence and the first and second unique index nucleotide sequences. A different combination of first and second barcoded index sequences can be used for each of the plurality of samples. The contacted samples can be pooled prior to amplifying. The identifying nucleotide sequence can comprise SEQ ID NO:1 or a sequence set forth in Table 1. The first barcoded index primer can be selected from SEQ ID NO:204-SEQ ID NO:233. The second barcoded index primer can be selected from SEQ ID NO:234-SEQ ID NO:253. The method can further comprise adding a linker to an affinity reagent to form the modified affinity reagent, wherein the linker comprises the identifying nucleotide sequence flanked on each end by an amplifying nucleotide sequence. The affinity reagent can be an antibody or an aptamer. The affinity reagent can be an antibody, wherein the adding step further comprises adding a linker to a region of the antibody that is not an antigen binding region. The affinity reagent can be an antibody, wherein the adding step further comprises adding a linker to a fragment crystallizable region (Fc region) of the antibody. The identifying nucleotide sequence (e.g., of the linker sequence) can have a length of about 10 nucleotides to about 20 nucleotides. The first amplifying sequence can comprise SEQ ID NO:2, and the second amplifying sequence can comprise SEQ ID NO:3. The linker can further comprise a fluorescent protein or a cleavable protein photocrosslinker.

In a further aspect, provided herein is a kit for high throughput multiplex protein quantification, comprising X modified affinity reagent(s) and Y pairs of barcoded index sequences wherein: X is equal to or greater than 1; Y is equal to or greater than 1; each modified affinity reagent comprising a linker, the linker comprising an identifying nucleotide sequence flanked by a pair of amplifying nucleotide sequences; each modified affinity reagent comprising a different identifying nucleotide sequence from other modified affinity reagents; and each pair of barcoded index primers comprises a unique combination of first and second barcoded index primers, wherein the first barcoded index primer comprises a universal sequence A, a first unique index nucleotide sequence, and a sequence configured to anneal to the first amplifying nucleotide sequence, and wherein the second barcoded index primer comprise a universal sequence B, a second unique index nucleotide sequence, and a sequence configured to anneal to the second amplifying nucleotide sequence. The linker can be selected from SEQ ID Nos:104-203. The first and second barcoded index primers can be selected from Table 3.

### BRIEF DESCRIPTION OF THE DRAWINGS

The present disclosure will be better understood and features, aspects, and advantages other than those set forth above will become apparent when consideration is given to the following detailed description thereof. Such detailed description makes reference to the following drawings, wherein:

FIG. 1 is a schematic illustrating an embodiment of dual index barcode analysis of in-solution DNA-barcoded protein arrays.

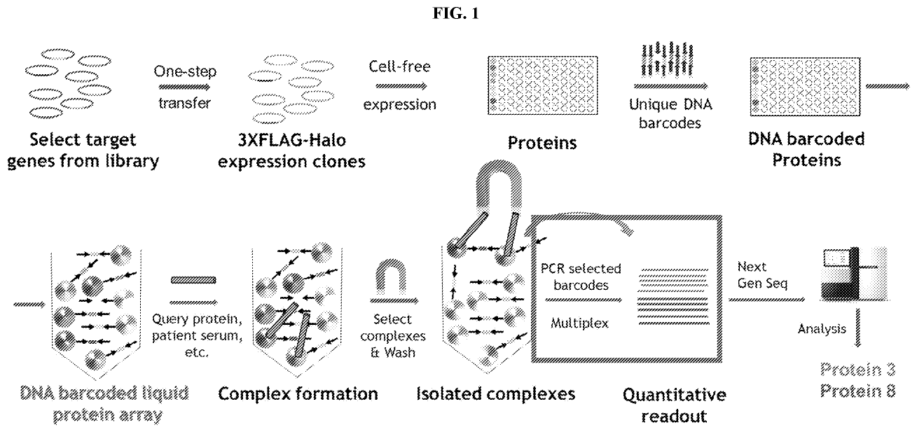

FIG. 2 is a schematic illustrating exemplary components of multiplex sequencing indexes.

FIG. 3 presents images of DNA gels showing the enrichment of antibodies in disease positive sera following amplification with different combinations of dual index barcode primers.

FIG. 4 presents a DNA agarose gel showing PCR reactions for four samples (HPV Positive 1-3 and HPV negative 4-5 serum samples incubated with the barcoded protein library) after adding unique dual index barcodes.

FIG. 5 presents a schematic illustrating an exemplary work flow for multiplexed detection methods of this disclosure.

### DETAILED DESCRIPTION

All publications, including but not limited to patents and patent applications, cited in this specification are herein incorporated by reference as though set forth in their entirety in the present application.

The compositions and methods described herein are based at least in part on the inventors' development of dual barcode indexes which allow for simultaneous analysis of 100s to 1000s of samples of interest and their interaction with 100s or more of proteins. As described herein, the technology exploits the ability of antibodies (or virtually any affinity reagent) to recognize their targets and the ability of unique DNA barcodes to enable detection of the antibodies and other affinity reagents using, for example, next generation DNA sequencing methods.

The inventors previously developed a strategy to uniquely barcode hundreds of proteins using a 12-bp DNA sequence, thereby producing an in-solution DNA-barcoded protein library. See U.S. Pat. No. 9,938,523, which is incorporated herein by reference in its entirety. By incubating this protein library with a “sample of interest” (e.g., other proteins, drugs, patient samples), the strategy permitted the identification of novel protein-protein interactions, immune responses, and other biological processes of interest using next generation sequencing (NGS). The compositions and methods of this disclosure solve the problem of how to multiplex the “sample of interest” and achieve simultaneous analysis of numerous targets. As described herein, the methods comprise adding, in a single step, unique index barcodes via polymerase chain reaction. Consequently, advantages of the presently described methods and compositions and methods are multifold and include, for example, the ability to assay a large number of samples of interest against hundreds of targets in a single next generation sequencing run, thereby increasing the high throughput capacity of the DNA barcoded protein array and lowering the cost of the array. The methods of this disclosure also reduce sample processing time since they do not require the multiple PCR cycles and sequence adaptor ligation reactions required by conventional protocols for multiplex detection.

Accordingly, in a first aspect, provided herein is a composition comprising a dual barcode index. As used herein, the term “dual barcode index” refers to a combination of two sets of unique nucleic acid barcodes. One set comprises unique DNA barcodes affixed to a plurality of proteins to form a DNA-barcoded protein library. The second set is a different set of unique DNA barcodes used to identify individual samples of interest when multiple samples are combined. When the protein library, barcoded with the first set of DNA barcodes, is contacted to a sample of interest, the first set of DNA barcodes permits identification of a variety of biomolecular interactions (e.g., evidence in the sample of a subject's immune response) by next generation sequencing. However, by adding the second set of DNA barcodes by polymerase chain reaction, it is possible to identify these unique biomolecular interactions in a given sample even when numerous samples are combined. Without the second set of DNA barcodes, it would be impossible to distinguish biomolecular interactions associated with a particular sample when multiple samples are combined. Accordingly, the dual barcode index is particularly advantageous for assaying a large number of samples of interest against hundreds of targets in a single next generation sequencing run, thereby increasing the high throughput capacity of each DNA barcoded protein array.

In some cases, the dual barcode index comprises a first set of DNA barcodes and a second set of DNA barcodes. As used herein, the term “barcode” refers to a known nucleic acid sequence that allows some feature of a nucleic acid with which the barcode is associated to be identified. In some cases, a barcode is flanked at its 5′ and 3′ ends by a set of common sequences (“flanking sequence”). In certain embodiments, the barcodes are DNA barcodes. For example, DNA barcodes of the first set comprise a nucleotide sequence of GCTGTACGGATT (SEQ ID NO:1) and/or nucleotide sequences set forth in Table 1. In some embodiments, each barcode sequence of Table 1 is flanked by a 5′ flanking sequence and a 3′ flanking sequence, thus forming the longer “linker” sequences, examples of which are set forth in Table 2, where DNA barcode sequences are shown in bold font. In some embodiments, the 5′ flanking sequence is (CCACCGCTGAGCAATAACTA; SEQ ID NO:2). In some embodiments, the 3′ flanking sequence is (CGTAGATGAGTCAACGGCCT; SEQ ID NO:3).

In some embodiments, the second set of DNA barcodes of the dual barcode index comprises nucleotide sequences set forth in Table 3. DNA barcodes of the second set are added to a DNA-barcoded protein array and function as forward and reverse primers for DNA amplification and sequencing. In this manner, DNA barcodes of the second set are referred to herein as “barcoded index primers.” In some embodiments, the barcoded index primers described herein are used in combination with affinity reagents comprising unique DNA barcodes as described in US Patent Pub. 2019/0366237, which is incorporated herein by reference in its entirety. As shown in Table 3, the forward barcoded index primers contain the 5′ flanking sequence (CCACCGCTGAGCAATAACTA; SEQ ID NO:2) of the first set of DNA barcodes, and the reverse barcoded index primers contain the 3′ flanking sequence (CGTAGATGAGTCAACGGCCT; SEQ ID NO:3) of the first set of DNA barcodes. A barcoded index primer may also comprise a universal sequence, which is a known sequence such as a particular sequencing adaptor required for next-generation sequencing.

The barcoded index primer sequences of this disclosure are exemplary only. It will be understood that other barcoded index primers and flanking sequences can be used with the dual barcoded index of this disclosure, provided that the barcoded index primer sequences are designed to anneal to the corresponding flanking sequence.

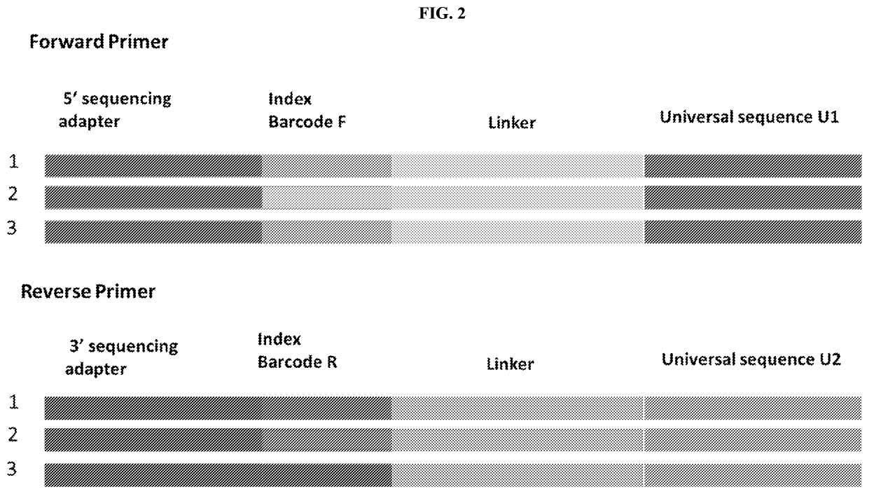

In some cases, barcoded index primers are added to a sample (e.g., biological sample, patient sample) to be contacted to the multiplex in-solution array of DNA barcoded proteins, and the sample-contacted array is amplified using any appropriate DNA amplification technique such as polymerase chain reaction (PCR). Preferably, the sample-contacted array is amplified using PCR. During DNA amplification, the barcoded index primers anneal to barcoded affinity reagents of a multiplex in-solution protein array and are amplified for multiplex analysis of many samples. Preferably, each dual barcode index comprises a different combination of DNA barcodes and sequence index primers, thereby reducing the number of unique sample identifiers needed for each reaction. For instance, referring to FIG. 2, the universal sequences U1 and U2 of the barcoded index primers can uniquely identify and anneal to the 5′ and 3′ flanking sequences (SEQ ID NO:2 and 3) on the in-solution DNA barcoded protein array. The index barcode regions of the forward and reverse sequences (n=9-12 base pairs) provide a unique identifier for the “sample of interest.” FIG. 2 illustrates an experiment involving nine samples of interest that have been contacted to the in-solution protein array to form target-affinity reagent complexes. To analyze all nine samples (N1 through N9) in a single NGS experiment, the samples are amplified in a single polymerase chain reaction step using different combinations of these constructs. For instance, the following combinations of forward and reverse DNA sequences can be used:

| | |
|---|---|
| Sample N1 | forward primer 1 and reverse primer 1 |
| Sample N2 | forward primer 1 and reverse primer 2 |
| Sample N3 | forward primer 1 and reverse primer 3 |
| Sample N4 | forward primer 2 and reverse primer 1 |
| Sample N5 | forward primer 2 and reverse primer 2 |
| Sample N6 | forward primer 2 and reverse primer 3 |
| Sample N7 | forward primer 3 and reverse primer 1 |
| Sample N8 | forward primer 3 and reverse primer 2 |
| sample N9 | forward primer 3 and reverse primer 3 |

This example demonstrates that six barcoded index primers (three forward and three reverse) can uniquely barcode and introduce sequencing adaptors for all nine samples. With this combination strategy, 10 barcoded forward primers and 10 barcoded reverse primers can introduce unique sequencing indexes for 100 biological samples, thus substantially increasing throughput of a single NGS experiment while reducing the cost of analysis of multiple samples.

TABLE 1
Exemplary Barcode Sequences

| | | Barcode |
|---|---|---|
| Barcode | | SEQ ID |
| name | DNA barcode sequence | NO: |
| Halo_BC1 | GTAGTGACAGGT | 4 |
| Halo_BC2 | TCTGTGAAGTCC | 5 |
| Halo_BC3 | ATCAGATCGCCT | 6 |
| Halo_BC4 | AATGTGGTCTCG | 7 |
| Halo_BC5 | CCTCTCCAAACA | 8 |
| Halo_BC6 | TACTGGACAAGG | 9 |
| Halo_BC7 | TATCGGAGTCCT | 10 |
| Halo_BC8 | GGTGGAGTTACT | 11 |
| Halo_BC9 | CGGCTACTATTG | 12 |
| Halo_BC10 | CCGAGCTATGTA | 13 |
| Halo_BC11 | ACTACGTCCAAC | 14 |
| Halo_BC12 | TTCATCCGAACG | 15 |
| Halo_BC13 | CGAAACGCTTAG | 16 |
| Halo_BC14 | GCCTAAGTTCCA | 17 |
| Halo_BC15 | CAATTCCCACGT | 18 |
| Halo_BC16 | CGGTGAGACATA | 19 |
| Halo_BC17 | CTCTGAGGTTTG | 20 |
| Halo_BC18 | TACTGTCACCCA | 21 |
| Halo_BC19 | CAGGAGGTACAT | 22 |
| Halo_BC20 | CTTCCTACAGCA | 23 |
| Halo_BC21 | TAGAAACCGAGG | 24 |
| Halo_BC22 | GAAAAGCGTACC | 25 |
| Halo_BC23 | CGCTCATAACTC | 26 |
| Halo_BC24 | GGCATATACGAC | 27 |
| Halo_BC25 | GTGCTCTATCAC | 28 |
| Halo_BC26 | GGAGCATTTCAC | 29 |
| Halo_BC27 | ATGGGTCTTCTG | 30 |
| Halo_BC28 | AAGTCCGTGAAC | 31 |
| Halo_BC29 | TGACATAGAGGG | 32 |
| Halo_BC30 | CGTCAATCGTGT | 33 |
| Halo_BC31 | GTTCGAAGCAAC | 34 |
| Halo_BC32 | ACCCGAATTCAC | 35 |
| Halo_BC33 | GAGGACTTCACA | 36 |
| Halo_BC34 | GATTCCACCGTA | 37 |
| Halo_BC35 | GTATTCGCCATG | 38 |
| Halo_BC36 | GCTTGTTATCCG | 39 |
| Halo_BC37 | CGTCCAACTATG | 40 |
| Halo_BC38 | GGTAACAGTGAC | 41 |
| Halo_BC39 | GCGCAAAAGAAG | 42 |
| Halo_BC40 | TGTGGTTGATCG | 43 |
| Halo_BC41 | TGTGGGATTGTG | 44 |
| Halo_BC42 | TGCTTCGGGATA | 45 |
| Halo_BC43 | GACAGCTCGTTA | 46 |
| Halo_BC44 | TAAGAAGCGCTC | 47 |
| Halo_BC45 | CATACACACTCC | 48 |
| Halo_BC46 | TGCCGCCAAAAT | 49 |
| Halo_BC47 | CGGACCTTCTAA | 50 |
| Halo_BC48 | TCTCACGTCAAC | 51 |
| Halo_BC49 | CGCAAGAGAACA | 52 |
| Halo_BC50 | TTAGCTTCCCTG | 53 |
| Halo_BC51 | GAAGCCAAGCAT | 54 |
| Halo_BC52 | TTCGTAGCGTGT | 55 |
| Halo_BC53 | GTCGCTGATCAA | 56 |
| Halo_BC54 | TCAACTGATCGG | 57 |
| Halo_BC55 | CCAGTTTCTACG | 58 |
| Halo_BC56 | ACCCATTGCGAT | 59 |
| Halo_BC57 | TCACCACCCTAT | 60 |
| Halo_BC58 | GGTCTTCACTTC | 61 |
| Halo_BC59 | GTTAGAGATGGG | 62 |
| Halo_BC60 | TCTTGCACACTC | 63 |
| Halo_BC61 | TTTTCTCTGCGG | 64 |
| Halo_BC62 | TCAGCCGAGTTA | 65 |
| Halo_BC63 | CTCGTGATCAGA | 66 |
| Halo_BC64 | CCTTTCTCGGAA | 67 |
| Halo_BC65 | ACGCTAGAGCTT | 68 |
| Halo_BC66 | TTCCCCGTTTAG | 69 |
| Halo_BC67 | AGAATCGCAACC | 70 |
| Halo_BC68 | GGAAGGAACTGT | 71 |
| Halo_BC69 | CTTGGCATCTTC | 72 |
| Halo_BC70 | AGGCCGATTTGT | 73 |
| Halo_BC71 | AACAAAGGGTCC | 74 |
| Halo_BC72 | CAATTGGTAGCC | 75 |
| Halo_BC73 | ACCATCGACTCA | 76 |
| Halo_BC74 | CGTGAGATGAAC | 77 |
| Halo_BC75 | CCATGGTCTTGT | 78 |
| Halo_BC76 | CAGATATGAGCGC | 79 |
| Halo_BC77 | GTGTGACAGAGT | 80 |
| Halo_BC78 | ATTGTGTGACGG | 81 |
| Halo_BC79 | CGGTAGTTTGCT | 82 |
| Halo_BC80 | GGACATGTCCAT | 83 |
| Halo_BC81 | TTGAGGGAGACA | 84 |
| Halo_BC82 | CGACATCCTCTA | 85 |
| Halo_BC83 | TGAGCGAGTTCA | 86 |
| Halo_BC84 | GACCTTCGGATT | 87 |
| Halo_BC85 | TGTAGATCCGCA | 88 |
| Halo_BC86 | TGGCACTCTAGA | 89 |
| Halo_BC87 | AACAGTAGTCGG | 90 |
| Halo_BC88 | TCATGCGGAAAG | 91 |
| Halo_BC89 | TCGAATCGTGTC | 92 |
| Halo_BC90 | GGTGTATAGCCA | 93 |
| Halo_BC91 | TTGCAGTGCAAG | 94 |
| Halo_BC92 | CGATTGCAGAAG | 95 |
| Halo_BC93 | CCAGACGTTGTT | 96 |
| Halo_BC94 | TGGTGGCCATAA | 97 |
| Halo_BC95 | CAGAGTCAATGG | 98 |
| Halo_BC96 | CCTATCATTCCC | 99 |
| Halo_BC97 | GAGGTATGACTC | 100 |
| Halo_BC98 | CTAGGTCAAGTC | 101 |
| Halo_BC99 | ACTCGGCTTTCA | 102 |
| Halo_BC10 | TTCACAAGCGGA | 103 |

TABLE 2
Exemplary Linker Sequences

| Name of | Linker: | |
|---|---|---|
| barcode | flanking seq- | |
| included in | barcode sequence- | |
| linker | flanking seq | SEQ ID NO: |
| Halo_BC1 | CCACCGCTGAGCAATAACTA | 104 |
| | GTAGTGACAGGT | |
| | CGTAGATGAGTCAACGGCCT | |
| Halo_BC2 | CCACCGCTGAGCAATAACTA | 105 |
| | TCTGTGAAGTCC | |
| | CGTAGATGAGTCAACGGCCT | |
| Halo_BC3 | CCACCGCTGAGCAATAACTA | 106 |
| | ATCAGATCGCCT | |
| | CGTAGATGAGTCAACGGCCT | |
| Halo_BC4 | CCACCGCTGAGCAATAACTA | 107 |
| | AATGTGGTCTCG | |
| | CGTAGATGAGTCAACGGCCT | |
| Halo_BC5 | CCACCGCTGAGCAATAACTA | 108 |
| | CCTCTCCAAACA | |
| | CGTAGATGAGTCAACGGCCT | |
| Halo_BC6 | CCACCGCTGAGCAATAACTA | 109 |
| | TACTGGACAAGG | |
| | CGTAGATGAGTCAACGGCCT | |
| Halo_BC7 | CCACCGCTGAGCAATAACTA | 110 |
| | TATCGGAGTCCT | |
| | CGTAGATGAGTCAACGGCCT | |
| Halo_BC8 | CCACCGCTGAGCAATAACTA | 111 |
| | GGTGGAGTTACT | |
| | CGTAGATGAGTCAACGGCCT | |
| Halo_BC9 | CCACCGCTGAGCAATAACTA | 112 |
| | CGGCTACTATTG | |
| | CGTAGATGAGTCAACGGCCT | |
| Halo_BC10 | CCACCGCTGAGCAATAACTA | 113 |
| | CCGAGCTATGTA | |
| | CGTAGATGAGTCAACGGCCT | |
| Halo_BC11 | CCACCGCTGAGCAATAACTA | 114 |
| | ACTACGTCCAAC | |
| | CGTAGATGAGTCAACGGCCT | |
| Halo_BC12 | CCACCGCTGAGCAATAACTA | 115 |
| | TTCATCCGAACG | |
| | CGTAGATGAGTCAACGGCCT | |
| Halo_BC13 | CCACCGCTGAGCAATAACTA | 116 |
| | CGAAACGCTTAG | |
| | CGTAGATGAGTCAACGGCCT | |
| Halo_BC14 | CCACCGCTGAGCAATAACTA | 117 |
| | GCCTAAGTTCCA | |
| | CGTAGATGAGTCAACGGCCT | |
| Halo_BC15 | CCACCGCTGAGCAATAACTA | 118 |
| | CAATTCCCACGT | |
| | CGTAGATGAGTCAACGGCCT | |
| Halo_BC16 | CCACCGCTGAGCAATAACTA | 119 |
| | CGGTGAGACATA | |
| | CGTAGATGAGTCAACGGCCT | |
| Halo_BC17 | CCACCGCTGAGCAATAACTA | 120 |
| | CTCTGAGGTTTG | |
| | CGTAGATGAGTCAACGGCCT | |
| Halo_BC18 | CCACCGCTGAGCAATAACTA | 121 |
| | TACTGTCACCCA | |
| | CGTAGATGAGTCAACGGCCT | |
| Halo_BC19 | CCACCGCTGAGCAATAACTA | 122 |
| | CAGGAGGTACAT | |
| | CGTAGATGAGTCAACGGCCT | |
| Halo_BC20 | CCACCGCTGAGCAATAACTA | 123 |
| | CTTCCTACAGCA | |
| | CGTAGATGAGTCAACGGCCT | |
| Halo_BC21 | CCACCGCTGAGCAATAACTA | 124 |
| | TAGAAACCGAGG | |
| | CGTAGATGAGTCAACGGCCT | |
| Halo_BC22 | CCACCGCTGAGCAATAACTA | 125 |
| | GAAAAGCGTACC | |
| | CGTAGATGAGTCAACGGCCT | |
| Halo_BC23 | CCACCGCTGAGCAATAACTA | 126 |
| | CGCTCATAACTC | |
| | CGTAGATGAGTCAACGGCCT | |
| Halo_BC24 | CCACCGCTGAGCAATAACTA | 127 |
| | GGCATATACGAC | |
| | CGTAGATGAGTCAACGGCCT | |
| Halo_BC25 | CCACCGCTGAGCAATAACTA | 128 |
| | GTGCTCTATCAC | |
| | CGTAGATGAGTCAACGGCCT | |
| Halo_BC26 | CCACCGCTGAGCAATAACTA | 129 |
| | GGAGCATTTCAC | |
| | CGTAGATGAGTCAACGGCCT | |
| Halo_BC27 | CCACCGCTGAGCAATAACTA | 130 |
| | ATGGGTCTTCTG | |
| | CGTAGATGAGTCAACGGCCT | |
| Halo_BC28 | CCACCGCTGAGCAATAACTA | 131 |
| | AAGTCCGTGAAC | |
| | CGTAGATGAGTCAACGGCCT | |
| Halo_BC29 | CCACCGCTGAGCAATAACTA | 132 |
| | TGACATAGAGGG | |
| | CGTAGATGAGTCAACGGCCT | |
| Halo_BC30 | CCACCGCTGAGCAATAACTA | 133 |
| | CGTCAATCGTGT | |
| | CGTAGATGAGTCAACGGCCT | |
| Halo_BC31 | CCACCGCTGAGCAATAACTA | 134 |
| | GTTCGAAGCAAC | |
| | CGTAGATGAGTCAACGGCCT | |
| Halo_BC32 | CCACCGCTGAGCAATAACTA | 135 |
| | ACCCGAATTCAC | |
| | CGTAGATGAGTCAACGGCCT | |
| Halo_BC33 | CCACCGCTGAGCAATAACTA | 136 |
| | GAGGACTTCACA | |
| | CGTAGATGAGTCAACGGCCT | |
| Halo_BC34 | CCACCGCTGAGCAATAACTA | 137 |
| | GATTCCACCGTA | |
| | CGTAGATGAGTCAACGGCCT | |
| Halo_BC35 | CCACCGCTGAGCAATAACTA | 138 |
| | GTATTCGCCATG | |
| | CGTAGATGAGTCAACGGCCT | |
| Halo_BC36 | CCACCGCTGAGCAATAACTA | 139 |
| | GCTTGTTATCCG | |
| | CGTAGATGAGTCAACGGCCT | |
| Halo_BC37 | CCACCGCTGAGCAATAACTA | 140 |
| | CGTCCAACTATG | |
| | CGTAGATGAGTCAACGGCCT | |
| Halo_BC38 | CCACCGCTGAGCAATAACTA | 141 |
| | GGTAACAGTGAC | |
| | CGTAGATGAGTCAACGGCCT | |
| Halo_BC39 | CCACCGCTGAGCAATAACTA | 142 |
| | GCGCAAAAGAAG | |
| | CGTAGATGAGTCAACGGCCT | |
| Halo_BC40 | CCACCGCTGAGCAATAACTA | 143 |
| | TGTGGTTGATCG | |
| | CGTAGATGAGTCAACGGCCT | |
| Halo_BC41 | CCACCGCTGAGCAATAACTA | 144 |
| | TGTGGGATTGTG | |
| | CGTAGATGAGTCAACGGCCT | |
| Halo_BC42 | CCACCGCTGAGCAATAACTA | 145 |
| | TGCTTCGGGATA | |
| | CGTAGATGAGTCAACGGCCT | |
| Halo_BC43 | CCACCGCTGAGCAATAACTA | 146 |
| | GACAGCTCGTTA | |
| | CGTAGATGAGTCAACGGCCT | |
| Halo_BC44 | CCACCGCTGAGCAATAACTA | 147 |
| | TAAGAAGCGCTC | |
| | CGTAGATGAGTCAACGGCCT | |
| Halo_BC45 | CCACCGCTGAGCAATAACTA | 148 |
| | CATACACACTCC | |
| | CGTAGATGAGTCAACGGCCT | |
| Halo_BC46 | CCACCGCTGAGCAATAACTA | 149 |
| | TGCCGCCAAAAT | |
| | CGTAGATGAGTCAACGGCCT | |
| Halo_BC47 | CCACCGCTGAGCAATAACTA | 150 |
| | CGGACCTTCTAA | |
| | CGTAGATGAGTCAACGGCCT | |
| Halo_BC48 | CCACCGCTGAGCAATAACTA | 151 |
| | TCTCACGTCAAC | |
| | CGTAGATGAGTCAACGGCCT | |
| Halo_BC49 | CCACCGCTGAGCAATAACTA | 152 |
| | CGCAAGAGAACA | |
| | CGTAGATGAGTCAACGGCCT | |
| Halo_BC50 | CCACCGCTGAGCAATAACTA | 153 |
| | TTAGCTTCCCTG | |
| | CGTAGATGAGTCAACGGCCT | |
| Halo_BC51 | CCACCGCTGAGCAATAACTA | 154 |
| | GAAGCCAAGCAT | |
| | CGTAGATGAGTCAACGGCCT | |
| Halo_BC52 | CCACCGCTGAGCAATAACTA | 155 |
| | TTCGTAGCGTGT | |
| | CGTAGATGAGTCAACGGCCT | |
| Halo_BC53 | CCACCGCTGAGCAATAACTA | 156 |
| | GTCGCTGATCAA | |
| | CGTAGATGAGTCAACGGCCT | |
| Halo_BC54 | CCACCGCTGAGCAATAACTA | 157 |
| | TCAACTGATCGG | |
| | CGTAGATGAGTCAACGGCCT | |
| Halo_BC55 | CCACCGCTGAGCAATAACTA | 158 |
| | CCAGTTTCTACG | |
| | CGTAGATGAGTCAACGGCCT | |
| Halo_BC56 | CCACCGCTGAGCAATAACTA | 159 |
| | ACCCATTGCGAT | |
| | CGTAGATGAGTCAACGGCCT | |
| Halo_BC57 | CCACCGCTGAGCAATAACTA | 160 |
| | TCACCACCCTAT | |
| | CGTAGATGAGTCAACGGCCT | |
| Halo_BC58 | CCACCGCTGAGCAATAACTA | 161 |
| | GGTCTTCACTTC | |
| | CGTAGATGAGTCAACGGCCT | |
| Halo_BC59 | CCACCGCTGAGCAATAACTA | 162 |
| | GTTAGAGATGGG | |
| | CGTAGATGAGTCAACGGCCT | |
| Halo_BC60 | CCACCGCTGAGCAATAACTA | 163 |
| | TCTTGCACACTC | |
| | CGTAGATGAGTCAACGGCCT | |
| Halo_BC61 | CCACCGCTGAGCAATAACTA | 164 |
| | TTTTCTCTGCGG | |
| | CGTAGATGAGTCAACGGCCT | |
| Halo_BC62 | CCACCGCTGAGCAATAACTA | 165 |
| | TCAGCCGAGTTA | |
| | CGTAGATGAGTCAACGGCCT | |
| Halo_BC63 | CCACCGCTGAGCAATAACTA | 166 |
| | CTCGTGATCAGA | |
| | CGTAGATGAGTCAACGGCCT | |
| Halo_BC64 | CCACCGCTGAGCAATAACTA | 167 |
| | CCTTTCTCGGAA | |
| | CGTAGATGAGTCAACGGCCT | |
| Halo_BC65 | CCACCGCTGAGCAATAACTA | 168 |
| | ACGCTAGAGCTT | |
| | CGTAGATGAGTCAACGGCCT | |
| Halo_BC66 | CCACCGCTGAGCAATAACTA | 169 |
| | TTCCCCGTTTAG | |
| | CGTAGATGAGTCAACGGCCT | |
| Halo_BC67 | CCACCGCTGAGCAATAACTA | 170 |
| | AGAATCGCAACC | |
| | CGTAGATGAGTCAACGGCCT | |
| Halo_BC68 | CCACCGCTGAGCAATAACTA | 171 |
| | GGAAGGAACTGT | |
| | CGTAGATGAGTCAACGGCCT | |
| Halo_BC69 | CCACCGCTGAGCAATAACTA | 172 |
| | CTTGGCATCTTC | |
| | CGTAGATGAGTCAACGGCCT | |
| Halo_BC70 | CCACCGCTGAGCAATAACTA | 173 |
| | AGGCCGATTTGT | |
| | CGTAGATGAGTCAACGGCCT | |
| Halo_BC71 | CCACCGCTGAGCAATAACTA | 174 |
| | AACAAAGGGTCC | |
| | CGTAGATGAGTCAACGGCCT | |
| Halo_BC72 | CCACCGCTGAGCAATAACTA | 175 |
| | CAATTGGTAGCC | |
| | CGTAGATGAGTCAACGGCCT | |
| Halo_BC73 | CCACCGCTGAGCAATAACTA | 176 |
| | ACCATCGACTCA | |
| | CGTAGATGAGTCAACGGCCT | |
| Halo_BC74 | CCACCGCTGAGCAATAACTA | 177 |
| | CGTGAGATGAAC | |
| | CGTAGATGAGTCAACGGCCT | |
| Halo_BC75 | CCACCGCTGAGCAATAACTA | 178 |
| | CCATGGTCTTGT | |
| | CGTAGATGAGTCAACGGCCT | |
| Halo_BC76 | CCACCGCTGAGCAATAACTA | 179 |
| | AGATATGAGCGC | |
| | CGTAGATGAGTCAACGGCCT | |
| Halo_BC77 | CCACCGCTGAGCAATAACTA | 180 |
| | GTGTGACAGAGT | |
| | CGTAGATGAGTCAACGGCCT | |
| Halo_BC78 | CCACCGCTGAGCAATAACTA | 181 |
| | ATTGTGTGACGG | |
| | CGTAGATGAGTCAACGGCCT | |
| Halo_BC79 | CCACCGCTGAGCAATAACTA | 182 |
| | CGGTAGTTTGCT | |
| | CGTAGATGAGTCAACGGCCT | |
| Halo_BC80 | CCACCGCTGAGCAATAACTA | 183 |
| | GGACATGTCCAT | |
| | CGTAGATGAGTCAACGGCCT | |
| Halo_BC81 | CCACCGCTGAGCAATAACTA | 184 |
| | TTGAGGGAGACA | |
| | CGTAGATGAGTCAACGGCCT | |
| Halo_BC82 | CCACCGCTGAGCAATAACTA | 185 |
| | CGACATCCTCTA | |
| | CGTAGATGAGTCAACGGCCT | |
| Halo_BC83 | CCACCGCTGAGCAATAACTA | 186 |
| | TGAGCGAGTTCA | |
| | CGTAGATGAGTCAACGGCCT | |
| Halo_BC84 | CCACCGCTGAGCAATAACTA | 187 |
| | GACCTTCGGATT | |
| | CGTAGATGAGTCAACGGCCT | |
| Halo_BC85 | CCACCGCTGAGCAATAACTA | 188 |
| | TGTAGATCCGCA | |
| | CGTAGATGAGTCAACGGCCT | |
| Halo_BC86 | CCACCGCTGAGCAATAACTA | 189 |
| | TGGCACTCTAGA | |
| | CGTAGATGAGTCAACGGCCT | |
| Halo_BC87 | CCACCGCTGAGCAATAACTA | 190 |
| | AACAGTAGTCGG | |
| | CGTAGATGAGTCAACGGCCT | |
| Halo_BC88 | CCACCGCTGAGCAATAACTA | 191 |
| | TCATGCGGAAAG | |
| | CGTAGATGAGTCAACGGCCT | |
| Halo_BC89 | CCACCGCTGAGCAATAACTA | 192 |
| | TCGAATCGTGTC | |
| | CGTAGATGAGTCAACGGCCT | |
| Halo_BC90 | CCACCGCTGAGCAATAACTA | 193 |
| | GGTGTATAGCCA | |
| | CGTAGATGAGTCAACGGCCT | |
| Halo_BC91 | CCACCGCTGAGCAATAACTA | 194 |
| | TTGCAGTGCAAG | |
| | CGTAGATGAGTCAACGGCCT | |
| Halo_BC92 | CCACCGCTGAGCAATAACTA | 195 |
| | CGATTGCAGAAG | |
| | CGTAGATGAGTCAACGGCCT | |
| Halo_BC93 | CCACCGCTGAGCAATAACTA | 196 |
| | CCAGACGTTGTT | |
| | CGTAGATGAGTCAACGGCCT | |
| Halo_BC94 | CCACCGCTGAGCAATAACTA | 197 |
| | TGGTGGCCATAA | |
| | CGTAGATGAGTCAACGGCCT | |
| Halo_BC95 | CCACCGCTGAGCAATAACTA | 198 |
| | CAGAGTCAATGG | |
| | CGTAGATGAGTCAACGGCCT | |
| Halo_BC96 | CCACCGCTGAGCAATAACTA | 199 |
| | CCTATCATTCCC | |
| | CGTAGATGAGTCAACGGCCT | |
| Halo_BC97 | CCACCGCTGAGCAATAACTA | 200 |
| | GAGGTATGACTC | |
| | CGTAGATGAGTCAACGGCCT | |
| Halo_BC98 | CCACCGCTGAGCAATAACTA | 201 |
| | CTAGGTCAAGTC | |
| | CGTAGATGAGTCAACGGCCT | |
| Halo_BC99 | CCACCGCTGAGCAATAACTA | 202 |
| | ACTCGGCTTTCA | |
| | CGTAGATGAGTCAACGGCCT | |
| Halo_BC100 | CCACCGCTGAGCAATAACTA | 203 |
| | TTCACAAGCGGA | |
| | CGTAGATGAGTCAACGGCCT | |

TABLE 3
Dual Barcode Indexes

| | | SEQ |
|---|---|---|
| | | ID |
| | | NO: |
| Forward | | |
| IndBCF1 | AATGATACGGCGACCACCGAGATCTACACGCT | 204 |
| | ATGATTGCGTCC TATGGTAATTGT AGGCCGTTGACTCA | |
| IndBCF2 | AATGATACGGCGACCACCGAGATCTACACGCT | 205 |
| | TGCTCATCGATG TATGGTAATTGT AGGCCGTTGACTCA | |
| IndBCF3 | AATGATACGGCGACCACCGAGATCTACACGCT | 206 |
| | CACAGGTTCTAC TATGGTAATTGT AGGCCGTTGACTCA | |
| IndBCF4 | AATGATACGGCGACCACCGAGATCTACACGCT | 207 |
| | CTGGCTTGATCT TATGGTAATTGT AGGCCGTTGACTCA | |
| IndBCF5 | AATGATACGGCGACCACCGAGATCTACACGCT | 208 |
| | TCTCTGTCCGAT TATGGTAATTGT AGGCCGTTGACTCA | |
| IndBCF6 | AATGATACGGCGACCACCGAGATCTACACGCT | 209 |
| | CAGCCATGGAAA TATGGTAATTGT AGGCCGTTGACTCA | |
| IndBCF7 | AATGATACGGCGACCACCGAGATCTACACGCT | 210 |
| | TATGTACCGGAG TATGGTAATTGT AGGCCGTTGACTCA | |
| IndBCF8 | AATGATACGGCGACCACCGAGATCTACACGCT | 211 |
| | ACTGTAACGCTC TATGGTAATTGT AGGCCGTTGACTCA | |
| IndBCF9 | AATGATACGGCGACCACCGAGATCTACACGCT | 212 |
| | CTAGCGTCCATT TATGGTAATTGT AGGCCGTTGACTCA | |
| IndBCF10 | AATGATACGGCGACCACCGAGATCTACACGCT | 213 |
| | TGGATATGCCGA TATGGTAATTGT AGGCCGTTGACTCA | |
| IndBCF11 | AATGATACGGCGACCACCGAGATCTACACGCT | 214 |
| | TTCCAACGTTGC TATGGTAATTGT AGGCCGTTGACTCA | |
| IndBCF12 | AATGATACGGCGACCACCGAGATCTACACGCT | 215 |
| | GGTGTGAACTCA TATGGTAATTGT AGGCCGTTGACTCA | |
| IndBCF13 | AATGATACGGCGACCACCGAGATCTACACGCT | 216 |
| | CAAAGGGAGATC TATGGTAATTGT AGGCCGTTGACTCA | |
| IndBCF14 | AATGATACGGCGACCACCGAGATCTACACGCT | 217 |
| | CTCACAATCCGT TATGGTAATTGT AGGCCGTTGACTCA | |
| IndBCF15 | AATGATACGGCGACCACCGAGATCTACACGCT | 218 |
| | GGTGGGTTTGAT TATGGTAATTGT AGGCCGTTGACTCA | |
| IndBCF16 | AATGATACGGCGACCACCGAGATCTACACGCT | 219 |
| | CCCTTTGTCTAG TATGGTAATTGT AGGCCGTTGACTCA | |
| IndBCF17 | AATGATACGGCGACCACCGAGATCTACACGCT | 220 |
| | TTTCTGCTGAGC TATGGTAATTGT AGGCCGTTGACTCA | |
| IndBCF18 | AATGATACGGCGACCACCGAGATCTACACGCT | 221 |
| | ACTTCTCCTGCT TATGGTAATTGT AGGCCGTTGACTCA | |
| IndBCF19 | AATGATACGGCGACCACCGAGATCTACACGCT | 222 |
| | CCGACCATAAGA TATGGTAATTGT AGGCCGTTGACTCA | |
| IndBCF20 | AATGATACGGCGACCACCGAGATCTACACGCT | 223 |
| | GACTGCTGATGA TATGGTAATTGT AGGCCGTTGACTCA | |
| IndBCF21 | AATGATACGGCGACCACCGAGATCTACACGCT | 224 |
| | AATCGAGGAGAG TATGGTAATTGT AGGCCGTTGACTCA | |
| IndBCF22 | AATGATACGGCGACCACCGAGATCTACACGCT | 225 |
| | AGCGCACTCTTT TATGGTAATTGT AGGCCGTTGACTCA | |
| IndBCF23 | AATGATACGGCGACCACCGAGATCTACACGCT | 226 |
| | AATTGGGTCGTC TATGGTAATTGT AGGCCGTTGACTCA | |
| IndBCF24 | AATGATACGGCGACCACCGAGATCTACACGCT | 227 |
| | TCGTTCGGACTA TATGGTAATTGT AGGCCGTTGACTCA | |
| IndBCF25 | AATGATACGGCGACCACCGAGATCTACACGCT | 228 |
| | AACGTAATCGCG TATGGTAATTGT AGGCCGTTGACTCA | |
| IndBCF26 | AATGATACGGCGACCACCGAGATCTACACGCT | 229 |
| | CATAGGAACGCT TATGGTAATTGT AGGCCGTTGACTCA | |
| IndBCF27 | AATGATACGGCGACCACCGAGATCTACACGCT | 230 |
| | GTCGACGCAAAT TATGGTAATTGT AGGCCGTTGACTCA | |
| IndBCF28 | AATGATACGGCGACCACCGAGATCTACACGCT | 231 |
| | TAAAGTCCTGGG TATGGTAATTGT AGGCCGTTGACTCA | |
| IndBCF29 | AATGATACGGCGACCACCGAGATCTACACGCT | 232 |
| | GCCGAACATACT TATGGTAATTGT AGGCCGTTGACTCA | |
| IndBCF30 | AATGATACGGCGACCACCGAGATCTACACGCT | 233 |
| | CGGATTGGTGTA TATGGTAATTGT AGGCCGTTGACTCA | |
| Reverse | | |
| IndBCR1 | CAAGCAGAAGACGGCATACGAGAT CTCCTTCATGAC | 234 |
| | AGTCAGCCAG CC CCACCGCTGAGCAAT | |
| IndBCR2 | CAAGCAGAAGACGGCATACGAGAT GAAGATCGATGG | 235 |
| | AGTCAGCCAG CC CCACCGCTGAGCAAT | |
| IndBCR3 | CAAGCAGAAGACGGCATACGAGAT AGGAACAGCGAT | 236 |
| | AGTCAGCCAG CC CCACCGCTGAGCAAT | |
| IndBCR4 | CAAGCAGAAGACGGCATACGAGAT CCAATCGATACG | 237 |
| | AGTCAGCCAG CC CCACCGCTGAGCAAT | |
| IndBCR5 | CAAGCAGAAGACGGCATACGAGAT ATCCAGGAGTTC | 238 |
| | AGTCAGCCAG CC CCACCGCTGAGCAAT | |
| IndBCR6 | CAAGCAGAAGACGGCATACGAGAT AACAAGCCGAAG | 239 |
| | AGTCAGCCAG CC CCACCGCTGAGCAAT | |
| IndBCR7 | CAAGCAGAAGACGGCATACGAGAT AGTGAGGCCATA | 240 |
| | AGTCAGCCAG CC CCACCGCTGAGCAAT | |
| IndBCR8 | CAAGCAGAAGACGGCATACGAGAT TAGACCCACTAG | 241 |
| | AGTCAGCCAG CC CCACCGCTGAGCAAT | |
| IndBCR9 | CAAGCAGAAGACGGCATACGAGAT TAGAGGTTGGGT | 242 |
| | AGTCAGCCAG CC CCACCGCTGAGCAAT | |
| IndBCR10 | CAAGCAGAAGACGGCATACGAGAT TCCCCTTCTACA | 243 |
| | AGTCAGCCAG CC CCACCGCTGAGCAAT | |
| IndBCR11 | CAAGCAGAAGACGGCATACGAGAT AATCCAACCCCT | 244 |
| | AGTCAGCCAG CC CCACCGCTGAGCAAT | |
| IndBCR12 | CAAGCAGAAGACGGCATACGAGAT GCTAAGGGTTGA | 245 |
| | AGTCAGCCAG CC CCACCGCTGAGCAAT | |
| IndBCR13 | CAAGCAGAAGACGGCATACGAGAT ACTGACGAGTCT | 246 |
| | AGTCAGCCAG CC CCACCGCTGAGCAAT | |
| IndBCR14 | CAAGCAGAAGACGGCATACGAGAT TGAGTTAGTGCG | 247 |
| | AGTCAGCCAG CC CCACCGCTGAGCAAT | |
| IndBCR15 | CAAGCAGAAGACGGCATACGAGAT GGTATACACGTG | 248 |
| | AGTCAGCCAG CC CCACCGCTGAGCAAT | |
| IndBCR16 | CAAGCAGAAGACGGCATACGAGAT CTAGGAGGTTCA | 249 |
| | AGTCAGCCAG CC CCACCGCTGAGCAAT | |
| IndBCR17 | CAAGCAGAAGACGGCATACGAGAT CGTTGTTCCTCT | 250 |
| | AGTCAGCCAG CC CCACCGCTGAGCAAT | |
| IndBCR18 | CAAGCAGAAGACGGCATACGAGAT CTTGTCCTCACA | 251 |
| | AGTCAGCCAG CC CCACCGCTGAGCAAT | |
| IndBCR19 | CAAGCAGAAGACGGCATACGAGAT GTCCAAAGCAAG | 252 |
| | AGTCAGCCAG CC CCACCGCTGAGCAAT | |
| IndBCR20 | CAAGCAGAAGACGGCATACGAGAT GAACACATGAGC | 253 |
| | AGTCAGCCAG CC CCACCGCTGAGCAAT | |

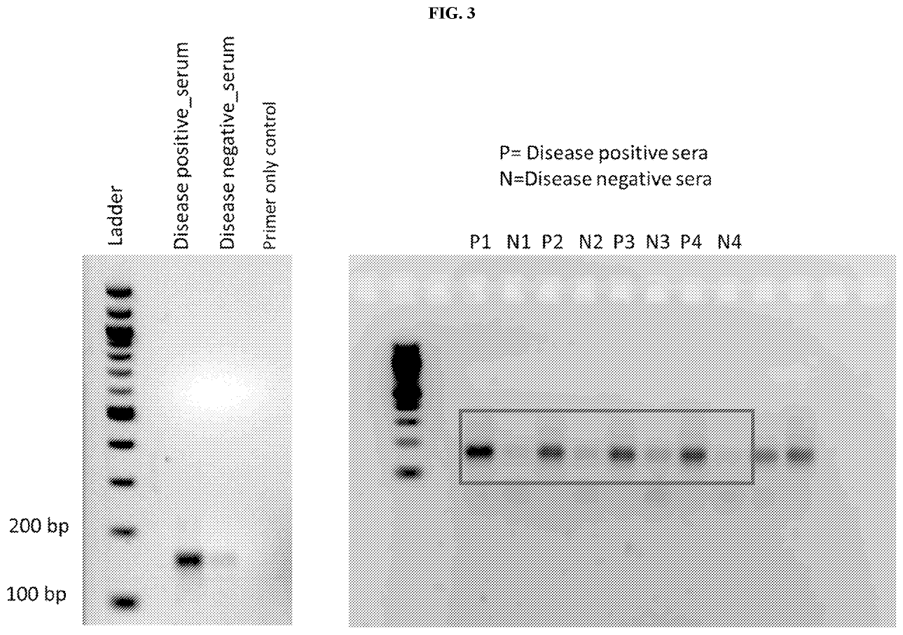

Referring to FIG. 3, analysis of positive patient samples (meaning the target of interest was detected in the sample) revealed stronger PCR bands as compared to negative samples when amplified with the dual barcode indexes of this disclosure. The DNA barcoded protein library (with HPV antigens) was incubated with patient serum samples (disease positive and negative) for 1 hour at room temperature. The time of incubation can vary from minimum of 30 min-24 hours. If incubated for longer periods, the assay can be performed at 4° C. Afterwards antigen-antibody complexes were isolated by adding protein G, Protein A/G or Protein L beads. Unbound reagent was washed away with washing buffer (1× Tris-buffered saline with 0.1-0.2% Tween 20 at pH 7.4). The enriched patient antibodies that formed complexes with DNA barcoded reagent were transferred into PCR plates (tubes). A unique forward and reverse dual barcode index combination primer pair was added to each patient pull down and was subjected to PCR/qPCR amplification. PCR products can be checked on a DNA gel and as shown in FIG. 3 clear differences can be seen between disease positive and disease negative sera for antibody enrichment.

In some cases, the DNA barcoded protein library is obtained according to the methods described in U.S. Pat. No. 9,938,523, which is incorporated herein by reference in its entirety.

As used herein, the term “affinity reagent” refers to an antibody, peptide, nucleic acid, aptamer, or other small molecule that specifically binds to a biological molecule (“biomolecule”) of interest in order to identify, track, capture, and/or influence its activity. In some embodiments, the affinity reagent is an antibody. In other embodiments, the affinity reagent is an aptamer. As described in US Patent Pub. 2019/0366237, incorporated herein by reference in its entirety, each affinity reagent (e.g., antibody) is chemically modified to add a linker that includes a unique DNA barcode, which is an identifying sequence flanked at its 5′ and 3′ ends by a set of common sequences (“flanking sequence”).

In some cases, the affinity reagents are antibodies having specificity for particular protein (e.g., antigen) targets, where the antibodies are linked to a DNA barcode. In such cases, an antibody affinity reagent is contacted to a sample under conditions that promote binding of the affinity reagent to its target antigen when present in said sample. Antibodies that are bound to their target antigens can be separated from unbound antibodies by washing unbound reagents from the sample. In some embodiments, the DNA barcode associated with the affinity reagent is amplified, such as by polymerase chain reaction (PCR), and the amplified barcode DNA is subjected to DNA sequencing to provide a measure of target antigen in the contacted sample.

Any antibody can be used for the affinity reagents of this disclosure. Preferably, the antibodies bind tightly (i.e., have high affinity for) target antigens. It will be understood that antibodies selected for use in affinity reagents will vary according to the particular application. In some cases, the antibodies have affinity for a particular protein only when in a certain conformation or having a specific modification.

In some embodiments, one or more modifications are made to the fragment crystallizable region (Fc region) of the affinity reagent antibody. The Fc region is the tail region of an antibody that interacts with cell surface receptors and some proteins of the complement system. In other embodiments, the modification is made to a common region far from the target binding region. In this manner, one may obtain a library of antibodies affinity reagents having specificity for desired targets, each antibody chemically modified to include a linked DNA barcode of known sequence. In certain embodiments, the DNA barcode sequence is flanked by common sequences.

In other embodiments, the affinity reagents are aptamers. The term “aptamer” as used herein refers to nucleic acids or peptide molecules that have affinity and bind specifically to a particular target. In particular, aptamers can comprise single-stranded (ss) oligonucleotides and peptides, including chemically synthesized peptides, that bind specifically to various biological molecules and are useful for in vitro or in vivo localization and quantification of various biological molecules. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Generally, nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues, and microorganisms.

Peptide aptamers are peptides selected or engineered to bind specific target molecules. These proteins consist of one or more peptide loops of variable sequence displayed by a protein scaffold. They can be isolated from combinatorial libraries and, in some cases, modified by directed mutation or rounds of variable region mutagenesis and selection. In vivo, peptide aptamers can bind cellular protein targets and exert biological effects, including interference with the normal protein interactions of their targeted molecules with other proteins. Libraries of peptide aptamers have been used as “mutagens,” in studies in which an investigator introduces a library that expresses different peptide aptamers into a cell population, selects for a desired phenotype, and identifies those aptamers associated with that phenotype.

Like antibody affinity reagents, aptamer affinity reagents comprise a linked DNA barcode sequence.

In some cases, the linker is a cleavable protein photocrosslinker, which can be photo-cleaved from the antibody or aptamer. In other cases, the linker is a ligand comprising a DNA barcode which can append to a target with a fusion tag. For example, the linker may be a Halo ligand comprising a barcode sequence appended to a Halo fusion tag. In other cases, the linker comprises a fluorescent probe in addition to the DNA barcode.

Methods

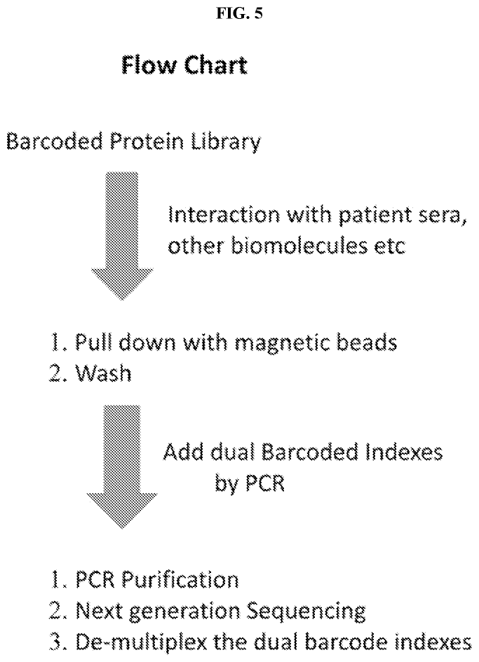

In another aspect, provided herein are methods for multiplexed detection and measurement of multiple targets in one or more samples using a single next-generation sequence run. FIG. 5 is a schematic illustrating an exemplary work flow for multiplexed detection methods of this disclosure. For instance, an in-solution barcoded protein array can be contacted to a biological sample obtained from a subject (e.g., patient sera) or any other sample comprising biomolecules. Complexes formed between the protein array and biomolecules in the sample are contacted to magnetic beads or a similar substrate for separating the complexes from solution. The separated sample is washed to remove non-specific binding. Index barcodes are then added by PCR. The PCR products are purified and subjected to next generation sequencing.

In some cases, the method for high throughput multiplex identification and quantification of target molecules in a plurality of samples comprises (a) for each of a plurality of samples, contacting the sample with a plurality of modified affinity reagents under conditions that promote binding of the modified affinity reagents to target molecules if present in the contacted sample, wherein each modified affinity reagent of the plurality comprises a unique identifying nucleotide sequence relative to other affinity reagents of the plurality, wherein each identifying nucleotide sequence is flanked by a first amplifying nucleotide sequence and a second amplifying nucleotide sequence; (b) contacting the contacted samples of step (a) to a first barcoded index primer and a second barcoded index primer under conditions that promote annealing of the first barcoded index primer and the second barcoded index primer to the first and second amplifying nucleotide sequences, wherein the first barcoded index primer comprises a universal sequence A, a first unique index nucleotide sequence, and a sequence configured to anneal to the first amplifying nucleotide sequence, and wherein the second barcoded index primer comprise a universal sequence B, a second unique index nucleotide sequence, and a sequence configured to anneal to the second amplifying nucleotide sequence; (c) amplifying the contacted samples of (b) to produce an amplified product; and (d) sequencing the amplified product whereby target molecules of each of the plurality of samples is identified and quantified based on detection of the identifying nucleotide sequence and the first and second unique index nucleotide sequences.

In some cases, the contacted samples are pooled. Using the forward and reverse multiplex index primers of this disclosure, it is possible to assay hundreds to thousands of samples of interest using amplification and sequencing such as by next-generation sequencing run. The methods of this disclosure are not limited to any particular sequencing platform; rather they are generally applicable and platform independent. Appropriate sequencing platforms for the methods of this disclosure include, without limitation, Illumina systems, Life Technologies Ion Torrent, and Qiagen GeneReader systems.

As used herein, a “sample” means any material that contains, or potentially contains, molecular targets associated with a particular disease or infectious agent. In some cases, the sample is any material that could be infected or contaminated by the presence of a pathogenic microorganism. Samples appropriate for use according to the methods provided herein include biological samples such as, for example, blood, plasma, serum, urine, saliva, tissues, cells, organs, organisms or portions thereof (e.g., mosquitoes, bacteria, plants or plant material), patient samples (e.g., feces or body fluids, such as urine, blood, serum, plasma, or cerebrospinal fluid), food samples, drinking water, and agricultural products. In some cases, samples appropriate for use according to the methods provided herein are “non-biological” in whole or in part. Non-biological samples include, without limitation, plastic and packaging materials, paper, clothing fibers, and metal surfaces. In certain embodiments, the methods provided herein are used to detect molecular targets associated with a particular disease or infectious agent on a surface or within a non-biological material that came in contact with, for example, a subject or a biological fluid or other material of a subject.

Any appropriate method can be used to detect and measure binding of affinity reagents to their targets in the sample. For example, PCR-based amplification can be performed directly on the sample following contacting to the modified affinity reagents. Exemplary methods of detection of PCR-based amplification products include: quantitative PCR (qPCR), visualizing DNA on an agarose gel with ethidium bromide (EtBr) staining, or other DNA fragment measuring approaches.

The terms “quantity”, “amount” and “level” are synonymous and generally well-understood in the art. The terms as used herein may particularly refer to an absolute quantification of a target molecule in a sample, or to a relative quantification of a target molecule in a sample, i.e., relative to another value such as relative to a reference value or to a range of values indicating a base-line expression of the biomarker. These values or ranges can be obtained from a single subject (e.g., human patient) or aggregated from a group of subjects. In some cases, target measurements are compared to a standard or set of standards.

In a further aspect, provided herein are methods for detecting and quantifying a subject's immune response to a disease (e.g., cancer, autoimmune disorder) or infectious agent such as a pathogenic microorganism. In such cases, affinity reagents are selected for their affinity for molecular targets associated with a particular disease or infectious agent. Advantageously, the affinity reagents described herein are well suited for multiplexed screening of a sample for many different infections. For example, one may assay a sample for many infections simultaneously to see which induced an immune response and to which infection-associated proteins triggered the response. For instance, DNA barcoded affinity reagents can be prepped for different subtypes of HPV (human papillomavirus) proteome and use it to look for early biomarkers for detection of HPV related cancers. In another application, DNA affinity reagents can be prepared for SARS-CoV2, and other corona virus proteomes to look at the global immune response among COVID-19 patients with different clinical symptoms. In general, these antigen libraries can be anything from proteomes of pathogens, proteins from cellular signaling pathways etc. Antigens of interest can be prepared by producing proteins in the cell free expression systems, bacterial, insect or mammalian expression systems. Halo ligand functionalized with unique DNA barcodes can be added into the expressed proteins to form covalent bonds with the Halo fusion tag. Barcoded proteins can be captured with anti-FLAG magnetic beads by utilizing the Flag tag in the expressed antigens. After washing the unbound proteins, excess barcodes etc, the DNA barcoded proteins/antigens can be eluted with excess amount of 3× Flag peptides. All eluted DNA barcoded proteins can be pooled together to produce the DNA-barcoded affinity reagent with a corresponding panel of proteins (100-300). The prepared DNA barcoded affinity reagent can be utilized for numerous downstream applications (immune response in patient sera, protein interactions, biomarkers, protein-drug interactions etc).

In certain embodiments, affinity reagents described herein are used to detect and, in some cases, monitor a subject's immune response to an infectious pathogen. By way of example, pathogens may comprise viruses including, without limitation, flaviruses, human immunodeficiency virus (HIV), Ebola virus, single stranded RNA viruses, single stranded DNA viruses, double-stranded RNA viruses, double-stranded DNA viruses. Other pathogens include but are not limited to parasites (e.g., malaria parasites and other protozoan and metazoan pathogens (Plasmodia species, Leishmania species, Schistosoma species, Trypanosoma species)), bacteria (e.g., Mycobacteria, in particular, M. tuberculosis, Salmonella, Streptococci, E. coli, Staphylococci), fungi (e.g., Candida species, Aspergillus species, Pneumocystis jirovecii and other Pneumocystis species), and prions. In some cases, the pathogenic microorganism, e.g. pathogenic bacteria, may be one which causes cancer in certain human cell types.

In certain embodiments, the methods detect human-pathogenic viruses (meaning viruses that cause human disease or pathology) including, without limitation, coronavirus (e.g., SARS-Cov-2), human immunodeficiency virus (HIV), Ebola virus, flaviviruses such Zika virus (e.g., Zika strain from the Americas, ZIKV), yellow fever virus, and dengue virus serotypes 1 (DENV1) and 3 (DENV3), and closely related viruses such as the chikungunya virus (CHIKV), HPV, and viruses of the family Caliciviridae (e.g., human enteric viruses such as norovirus and sapovirus).

The terms “detect” or “detection” as used herein indicate the determination of the existence, presence or fact of a target molecule in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent.

Articles of Manufacture

In another aspect, provided herein are articles of manufacture useful for multiplex detection of target molecules, including infection-associated or disease-associated molecules (e.g., cancer associated). In certain embodiments, the article of manufacture is a kit for high throughput multiplex protein quantification, comprising X modified affinity reagent(s) and Y pairs of barcoded index sequences wherein: X is equal to or greater than 1; Y is equal to or greater than 1; each modified affinity reagent comprising a linker, the linker comprising an identifying nucleotide sequence flanked by a pair of amplifying nucleotide sequences; each modified affinity reagent comprising a different identifying nucleotide sequence from other modified affinity reagents; and each pair of barcoded index sequences comprises a unique combination of first and second barcoded index sequences, wherein the first barcoded index sequence comprises a universal sequencing adaptor, a first unique index nucleotide sequence, and a sequence configured to anneal to the first amplifying nucleotide sequence, and wherein the second barcoded index sequence comprise a universal sequencing adaptor, a second unique index nucleotide sequence, and a sequence configured to anneal to the second amplifying nucleotide sequence. In some cases, the linker is selected from SEQ ID Nos:104-203. The first and second barcoded index sequences can be selected from Table 3. Optionally, a kit can further include instructions for performing the multiplex detection and/or amplification methods described herein.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

The indefinite articles “a” and “an,” as used herein in the specification and in the claims, unless clearly indicated to the contrary, should be understood to mean “at least one.”

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Unless otherwise indicated, any nucleic acid sequences are written left to right in 5′ to 3′ orientation; amino acid sequences are written left to right in amino to carboxy orientation, respectively.

Schematic flow charts included are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Examples

Materials and Methods

Proteins expressing different subtypes of the HPV proteomes were produced using the Thermo Fisher IVTT cell free expression system. 5 uL of each unique DNA barcode with common flanking regions was added to each of the antigens/proteins produced and allowed to form covalent bonds for 1 hour. After 1 hour, for each reaction, 50 ul bead slurry of anti-FLAG magnetic beads were added and incubated over-night at 4° C. with agitation (800 rpm) for 16 hours. Beads were washed 3 times to remove any unbound proteins and excess barcodes. DNA barcoded proteins were eluted with 100 uL of 500 nM 3× FLAG peptide elution buffer after incubating for two hours. Barcoded proteins/antigens were pooled into one container and aliquoted (50 uL each) and stored at −80°.

50 μL aliquot (or aliquots) of an in-solution barcoded protein array was taken out from the −80° C. freezer. This library was then mixed with 50 μL of 1:100 diluted (1×, Tris-Buffered Saline/Tween 20 buffer, pH 7.4) serum sample, query protein etc. The samples were added to a 96 deep well block and was incubated over-night at 4° C./950 rpm.

The required amount of protein A/G magnetic beads or query protein coated magnetic beads etc (20 μL of bead slurry per sample) was added to a micro centrifuge tube. The beads were washed with 3 bed volumes of 1×TBST (1× Tris-Buffered Saline with 1% Tween 20, pH 7.4). After each wash the tube was placed on a magnetic stand to collect the beads. Supernatant was removed and the washing step was repeated 3 times. After the final wash 25 vL of bead slurry in 1×TBST pH 7.4 was added to the samples in the deep well block. The plate was incubated at 4° C. for 3 hours at 950 rpm. After 3 hours the plate was placed on a magnetic plate stand. The supernatant was removed and the beads were gently washed with 300 μl of 1×TBST pH 7.4 three times followed by 3 washes with 1×TBS pH 7.4. After the final wash 150 μL of 1×TBS pH 7.4 was added, and the samples were boiled at 95° C. for 5 min and supernatant was stored at −20° C. until PCR amplification.

PCR Amplification with Dual Barcode Indexes.

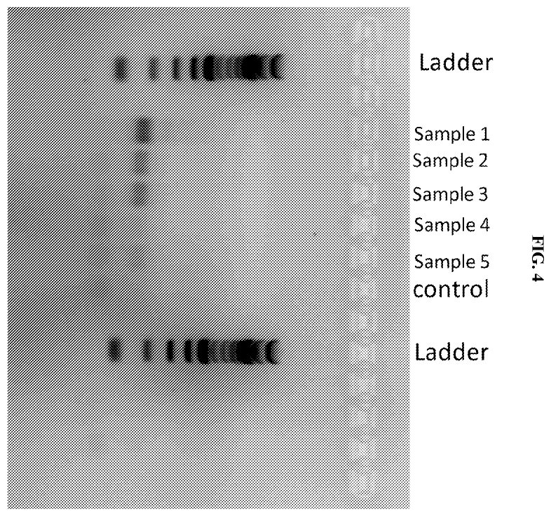

For 5 μl of the interacted sample unique dual index barcodes forward (IndBCF1, 2 etc dual index primer) and reverse (IndBCR1, 2 . . . etc) was added (0.5 μM final concentration) along with 25.00 μL of 2× Sapphire PCR mix and 18 μL of water in a PCR plate. Each sample has a unique combination of forward and reverse dual index barcodes. The PCR reaction was conducted for 15 cycles (initial step 1 min/94° C., denaturation 15 sec/98° C., 10 sec/60° C., extension 10 sec/72° C., pfinal extension 15 sec/72° C.). The PCR products were purified with PCR cleanup (Qiagen) and equal volumes of each dual index barcoded samples were pooled and subjected to next generation sequencing. Once the sequencing was complete, the samples were de-multiplexed and analyzed for enrichment. FIGS. 3 and 4 show amplification after adding unique dual sample indexes for various patient sample pulldowns (protein A/G beads) after interacting with the reagent. As shown in FIGS. 3 and 4 patient sera of HPV positive cancer patients showed a clear enrichment of antibody response whereas HPV negative patient samples showed only a weak background signal.

We claim:

1. A composition comprising
(i) a plurality of modified affinity reagents, each affinity reagent of the plurality comprising a unique identifying nucleotide sequence relative to other affinity reagents of the plurality, wherein each identifying nucleotide sequence is flanked by a first amplifying nucleotide sequence and a second amplifying nucleotide sequence;
(ii) a first barcoded index primer comprising a universal sequence A, a first unique index nucleotide sequence, and a sequence configured to anneal to the first amplifying nucleotide sequence; and
(iii) a second barcoded index sequence comprising a universal sequence B, a second unique index nucleotide sequence, and sequence configured to anneal to the second amplifying nucleotide sequence.

2. The composition of claim 1, wherein the first barcoded index primer is selected from SEQ ID NO:204-SEQ ID NO:233.

3. The composition of claim 1, wherein the second barcoded index primer is selected from SEQ ID NO:234-SEQ ID NO:253.

4. The composition of claim 1, wherein identifying nucleotide sequences are selected from SEQ ID NO:1 and barcode sequences set forth in Table 1.

5. The composition of claim 1, wherein affinity reagents of the plurality are antibodies.

6. The composition of claim 1, wherein affinity reagents of the plurality are peptide aptamers or nucleic acid aptamers.

7. The composition of claim 1, wherein an identifying nucleotide sequence is attached to an affinity reagent by a linker comprising (a) a cleavable protein photocrosslinker; or (b) a fluorescent moiety.

8. (canceled)

9. A method for high throughput multiplex identification and quantification of target molecules in a plurality of samples, comprising:
(a) for each of a plurality of samples, contacting the sample with a plurality of modified affinity reagents under conditions that promote binding of the modified affinity reagents to target molecules if present in the contacted sample, wherein each modified affinity reagent of the plurality comprises a unique identifying nucleotide sequence relative to other affinity reagents of the plurality, wherein each identifying nucleotide sequence is flanked by a first amplifying nucleotide sequence and a second amplifying nucleotide sequence;
(b) contacting the contacted samples of step (a) to a first barcoded index primer and a second barcoded index primer under conditions that promote annealing of the first barcoded index primer and the second barcoded index primer to the first and second amplifying nucleotide sequences,
wherein the first barcoded index primer comprises a universal sequence A, a first unique index nucleotide sequence, and a sequence configured to anneal to the first amplifying nucleotide sequence, and
wherein the second barcoded index primer comprises a universal sequence B, a second unique index nucleotide sequence, and a sequence configured to anneal to the second amplifying nucleotide sequence;
(c) amplifying the contacted samples of (b) to produce an amplified product; and
(d) sequencing the amplified product whereby target molecules of each of the plurality of samples is identified and quantified based on detection of the identifying nucleotide sequence and the first and second unique index nucleotide sequences.

10. The method of claim 9, wherein a different combination of first and second barcoded index sequences are used for each of the plurality of samples.

11. The method of claim 9, wherein the contacted samples are pooled prior to amplifying.

12. The method of claim 9, wherein the identifying nucleotide sequence comprises SEQ ID NO:1 or a sequence set forth in Table 1.

13. The method of claim 9, wherein the first barcoded index primer is selected from SEQ ID NO:204-SEQ ID NO:233.

14. The method of claim 9, wherein the second barcoded index primer is selected from SEQ ID NO:234-SEQ ID NO:253.

15. The method of claim 9, further comprising adding a linker to an affinity reagent to form the modified affinity reagent, wherein the linker comprises the identifying nucleotide sequence flanked on each end by an amplifying nucleotide sequence.

16. The method of claim 9, wherein the affinity reagent is an antibody or an aptamer.

17. The method of claim 16, wherein the affinity reagent is an antibody and wherein the adding step further comprises adding a linker to a region of the antibody that is not an antigen binding region.

18. The method of claim 16, wherein the affinity reagent is an antibody and wherein the adding step further comprises adding a linker to a fragment crystallizable region (Fc region) of the antibody.

19. (canceled)

20. The method of claim 19, wherein the first amplifying sequence comprises SEQ ID NO:2, and wherein the second amplifying sequence comprises SEQ ID NO:3.

21. (canceled)

22. A kit for high throughput multiplex protein quantification, comprising X modified affinity reagent(s) and Y pairs of barcoded index sequences wherein:
X is equal to or greater than 1;
Y is equal to or greater than 1;
each modified affinity reagent comprising a linker, the linker comprising an identifying nucleotide sequence flanked by a pair of amplifying nucleotide sequences;
each modified affinity reagent comprising a different identifying nucleotide sequence from other modified affinity reagents; and
each pair of barcoded index primers comprises a unique combination of first and second barcoded index primers, wherein the first barcoded index primer comprises a universal sequence A, a first unique index nucleotide sequence, and a sequence configured to anneal to the first amplifying nucleotide sequence, and wherein the second barcoded index primer comprise a universal sequence B, a second unique index nucleotide sequence, and a sequence configured to anneal to the second amplifying nucleotide sequence.

23. The kit of claim 22, wherein the linker is selected from SEQ ID Nos:104-203, and/or wherein the first and second barcoded index primers are selected from Table 3.

24. (canceled)


SEQUENCE LISTING (253 sequences)

SEQ ID NO: 1 - DNA, 12 bp, Artificial Sequence
  Synthetic DNA barcode
gctgtacggatt

SEQ ID NO: 2 - DNA, 20 bp, Artificial Sequence
  Synthetic- 5′ flanking sequence
ccaccgctgagcaataacta

SEQ ID NO: 3 - DNA, 20 bp, Artificial Sequence
  Synthetic- 3′ flanking sequence
ccaccgctgagcaataacta

SEQ ID NO: 4 - DNA, 12 bp, Artificial Sequence
  Synthetic- Halo-BC1
gtagtgacaggt

SEQ ID NO: 5 - DNA, 12 bp, Artificial Sequence
  Synthetic- Halo_BC2
tctgtgaagtcc

SEQ ID NO: 6 - DNA, 12 bp, Artificial Sequence
  Synthetic- Halo_BC3
atcagatcgcct

SEQ ID NO: 7 - DNA, 12 bp, Artificial Sequence
  Synthetic- Halo_BC4
aatgtggtctcg

SEQ ID NO: 8 - DNA, 12 bp, Artificial Sequence
  Synthetic- Halo_BC5
cctctccaaaca

SEQ ID NO: 9 - DNA, 12 bp, Artificial Sequence
  Synthetic- Halo_BC6
tactggacaagg

SEQ ID NO: 10 - DNA, 12 bp, Artificial Sequence
  Synthetic- Halo_BC7
tatcggagtcct

SEQ ID NO: 11 - DNA, 12 bp, Artificial Sequence
  Synthetic- Halo_BC8
ggtggagttact

SEQ ID NO: 12 - DNA, 12 bp, Artificial Sequence
  Synthetic- Halo_BC9
cggctactattg

SEQ ID NO: 13 - DNA, 12 bp, Artificial Sequence
  Synthetic- Halo_BC10
ccgagctatgta

SEQ ID NO: 14 - DNA, 12 bp, Artificial Sequence
  Synthetic- Halo_BC11
actacgtccaac

SEQ ID NO: 15 - DNA, 12 bp, Artificial Sequence
  Synthetic- Halo_BC12
ttcatccgaacg

SEQ ID NO: 16 - DNA, 12 bp, Artificial Sequence
  Synthetic- Halo_BC13
cgaaacgcttag

SEQ ID NO: 17 - DNA, 12 bp, Artificial Sequence
  Synthetic- Halo_BC14
gcctaagttcca

SEQ ID NO: 18 - DNA, 12 bp, Artificial Sequence
  Synthetic- Halo_BC15
caattcccacgt

SEQ ID NO: 19 - DNA, 12 bp, Artificial Sequence
  Synthetic- Halo_BC16
cggtgagacata

SEQ ID NO: 20 - DNA, 12 bp, Artificial Sequence
  Synthetic- Halo_BC17
ctctgaggtttg

SEQ ID NO: 21 - DNA, 12 bp, Artificial Sequence
  Synthetic- Halo_BC18
tactgtcaccca

SEQ ID NO: 22 - DNA, 12 bp, Artificial Sequence
  Synthetic- Halo_BC19
caggaggtacat

SEQ ID NO: 23 - DNA, 12 bp, Artificial Sequence
  Synthetic- Halo_BC20
cttcctacagca

SEQ ID NO: 24 - DNA, 12 bp, Artificial Sequence
  Synthetic- Halo_BC21
tagaaaccgagg

SEQ ID NO: 25 - DNA, 12 bp, Artificial Sequence
  Synthetic- Halo_BC22
gaaaagcgtacc

SEQ ID NO: 26 - DNA, 12 bp, Artificial Sequence
  Synthetic- Halo_BC23
cgctcataactc

SEQ ID NO: 27 - DNA, 12 bp, Artificial Sequence
  Synthetic- Halo_BC24
ggcatatacgac

SEQ ID NO: 28 - DNA, 12 bp, Artificial Sequence
  Synthetic- Halo_BC25
gtgctctatcac

SEQ ID NO: 29 - DNA, 12 bp, Artificial Sequence
  Synthetic- Halo_BC26
ggagcatttcac

SEQ ID NO: 30 - DNA, 12 bp, Artificial Sequence
  Synthetic- Halo_BC27
atgggtcttctg

SEQ ID NO: 31 - DNA, 12 bp, Artificial Sequence
  Synthetic- Halo_BC28
aagtccgtgaac

SEQ ID NO: 32 - DNA, 12 bp, Artificial Sequence
  Synthetic- Halo_BC29
tgacatagaggg

SEQ ID NO: 33 - DNA, 12 bp, Artificial Sequence
  Synthetic- Halo_BC30
cgtcaatcgtgt

SEQ ID NO: 34 - DNA, 12 bp, Artificial Sequence
  Synthetic- Halo_BC31
gttcgaagcaac

SEQ ID NO: 35 - DNA, 12 bp, Artificial Sequence
  Synthetic- Halo_BC32
acccgaattcac

SEQ ID NO: 36 - DNA, 12 bp, Artificial Sequence
  Synthetic- Halo_BC33
gaggacttcaca

SEQ ID NO: 37 - DNA, 12 bp, Artificial Sequence
  Synthetic- Halo_BC34
gattccaccgta

SEQ ID NO: 38 - DNA, 12 bp, Artificial Sequence
  Synthetic- Halo_BC35
gtattcgccatg

SEQ ID NO: 39 - DNA, 12 bp, Artificial Sequence
  Synthetic- Halo_BC36
gcttgttatccg

SEQ ID NO: 40 - DNA, 12 bp, Artificial Sequence
  Synthetic- Halo_BC37
cgtccaactatg

SEQ ID NO: 41 - DNA, 12 bp, Artificial Sequence
  Synthetic- Halo_BC38
ggtaacagtgac

SEQ ID NO: 42 - DNA, 12 bp, Artificial Sequence
  Synthetic- Halo_BC39
gcgcaaaagaag

SEQ ID NO: 43 - DNA, 12 bp, Artificial Sequence
  Synthetic- Halo_BC40
tgtggttgatcg

SEQ ID NO: 44 - DNA, 12 bp, Artificial Sequence
  Synthetic- Halo_BC41
tgtgggattgtg

SEQ ID NO: 45 - DNA, 12 bp, Artificial Sequence
  Synthetic- Halo_BC42
tgcttcgggata

SEQ ID NO: 46 - DNA, 12 bp, Artificial Sequence
  Synthetic- Halo_BC43
gacagctcgtta

SEQ ID NO: 47 - DNA, 12 bp, Artificial Sequence
  Synthetic- Halo_BC44
taagaagcgctc

SEQ ID NO: 48 - DNA, 12 bp, Artificial Sequence
  Synthetic- Halo_BC45
catacacactcc

SEQ ID NO: 49 - DNA, 12 bp, Artificial Sequence
  Synthetic- Halo_BC46
tgccgccaaaat

SEQ ID NO: 50 - DNA, 12 bp, Artificial Sequence
  Synthetic- Halo_BC47
cggaccttctaa

SEQ ID NO: 51 - DNA, 12 bp, Artificial Sequence
  Synthetic- Halo_BC48
tctcacgtcaac

SEQ ID NO: 52 - DNA, 12 bp, Artificial Sequence
  Synthetic- Halo_BC49
cgcaagagaaca

SEQ ID NO: 53 - DNA, 12 bp, Artificial Sequence
  Synthetic- Halo_BC50
ttagcttccctg

SEQ ID NO: 54 - DNA, 12 bp, Artificial Sequence
  Synthetic- Halo_BC51
gaagccaagcat

SEQ ID NO: 55 - DNA, 12 bp, Artificial Sequence
  Synthetic- Halo_BC52
ttcgtagcgtgt

SEQ ID NO: 56 - DNA, 12 bp, Artificial Sequence
  Synthetic- Halo_BC53
gtcgctgatcaa

SEQ ID NO: 57 - DNA, 12 bp, Artificial Sequence
  Synthetic- Halo_BC54
tcaactgatcgg

SEQ ID NO: 58 - DNA, 12 bp, Artificial Sequence
  Synthetic- Halo_BC55
ccagtttctacg

SEQ ID NO: 59 - DNA, 12 bp, Artificial Sequence
  Synthetic- Halo_BC56
acccattgcgat

SEQ ID NO: 60 - DNA, 12 bp, Artificial Sequence
  Synthetic- Halo_BC57
tcaccaccctat

SEQ ID NO: 61 - DNA, 12 bp, Artificial Sequence
  Synthetic- Halo_BC58
ggtcttcacttc

SEQ ID NO: 62 - DNA, 12 bp, Artificial Sequence
  Synthetic- Halo_BC59
gttagagatggg

SEQ ID NO: 63 - DNA, 12 bp, Artificial Sequence
  Synthetic- Halo_BC60
tcttgcacactc

SEQ ID NO: 64 - DNA, 12 bp, Artificial Sequence
  Synthetic- Halo_BC61
ttttctctgcgg

SEQ ID NO: 65 - DNA, 12 bp, Artificial Sequence
  Synthetic- Halo_BC62
tcagccgagtta

SEQ ID NO: 66 - DNA, 12 bp, Artificial Sequence
  Synthetic- Halo_BC63
ctcgtgatcaga

SEQ ID NO: 67 - DNA, 12 bp, Artificial Sequence
  Synthetic- Halo_BC64
cctttctcggaa

SEQ ID NO: 68 - DNA, 12 bp, Artificial Sequence
  Synthetic- Halo_BC65
acgctagagctt

SEQ ID NO: 69 - DNA, 12 bp, Artificial Sequence
  Synthetic- Halo_BC66
ttccccgtttag

SEQ ID NO: 70 - DNA, 12 bp, Artificial Sequence
  Synthetic- Halo_BC67
agaatcgcaacc

SEQ ID NO: 71 - DNA, 12 bp, Artificial Sequence
  Synthetic- Halo_BC68
ggaaggaactgt

SEQ ID NO: 72 - DNA, 12 bp, Artificial Sequence
  Synthetic- Halo_BC69
cttggcatcttc

SEQ ID NO: 73 - DNA, 12 bp, Artificial Sequence
  Synthetic- Halo_BC70
aggccgatttgt

SEQ ID NO: 74 - DNA, 12 bp, Artificial Sequence
  Synthetic- Halo_BC71
aacaaagggtcc

SEQ ID NO: 75 - DNA, 12 bp, Artificial Sequence
  Synthetic- Halo_BC72
caattggtagcc

SEQ ID NO: 76 - DNA, 12 bp, Artificial Sequence
  Synthetic- Halo_BC73
accatcgactca

SEQ ID NO: 77 - DNA, 12 bp, Artificial Sequence
  Synthetic- Halo_BC74
cgtgagatgaac

SEQ ID NO: 78 - DNA, 12 bp, Artificial Sequence
  Synthetic- Halo_BC75
ccatggtcttgt

SEQ ID NO: 79 - DNA, 13 bp, Artificial Sequence
  Synthetic- Halo_BC76
cagatatgagcgc

SEQ ID NO: 80 - DNA, 12 bp, Artificial Sequence
  Synthetic- Halo_BC77
gtgtgacagagt

SEQ ID NO: 81 - DNA, 12 bp, Artificial Sequence
  Synthetic- Halo_BC78
attgtgtgacgg

SEQ ID NO: 82 - DNA, 12 bp, Artificial Sequence
  Synthetic- Halo_BC79
cggtagtttgct

SEQ ID NO: 83 - DNA, 12 bp, Artificial Sequence
  Synthetic- Halo_BC80
ggacatgtccat

SEQ ID NO: 84 - DNA, 12 bp, Artificial Sequence
  Synthetic- Halo_BC81
ttgagggagaca

SEQ ID NO: 85 - DNA, 12 bp, Artificial Sequence
  Synthetic- Halo_BC82
cgacatcctcta

SEQ ID NO: 86 - DNA, 12 bp, Artificial Sequence
  Synthetic- Halo_BC83
tgagcgagttca

SEQ ID NO: 87 - DNA, 12 bp, Artificial Sequence
  Synthetic- Halo_BC84
gaccttcggatt

SEQ ID NO: 88 - DNA, 12 bp, Artificial Sequence
  Synthetic- Halo_BC85
tgtagatccgca

SEQ ID NO: 89 - DNA, 12 bp, Artificial Sequence
  Synthetic- Halo_BC86
tggcactctaga

SEQ ID NO: 90 - DNA, 12 bp, Artificial Sequence
  Synthetic- Halo_BC87
aacagtagtcgg

SEQ ID NO: 91 - DNA, 12 bp, Artificial Sequence
  Synthetic- Halo_BC88
tcatgcggaaag

SEQ ID NO: 92 - DNA, 12 bp, Artificial Sequence
  Synthetic- Halo_BC89
tcgaatcgtgtc

SEQ ID NO: 93 - DNA, 12 bp, Artificial Sequence
  Synthetic- Halo_BC90
ggtgtatagcca

SEQ ID NO: 94 - DNA, 12 bp, Artificial Sequence
  Synthetic- Halo_BC91
ttgcagtgcaag

SEQ ID NO: 95 - DNA, 12 bp, Artificial Sequence
  Synthetic- Halo_BC92
cgattgcagaag

SEQ ID NO: 96 - DNA, 12 bp, Artificial Sequence
  Synthetic- Halo_BC93
ccagacgttgtt

SEQ ID NO: 97 - DNA, 12 bp, Artificial Sequence
  Synthetic- Halo_BC94
tggtggccataa

SEQ ID NO: 98 - DNA, 12 bp, Artificial Sequence
  Synthetic- Halo_BC95
cagagtcaatgg

SEQ ID NO: 99 - DNA, 12 bp, Artificial Sequence
  Synthetic- Halo_BC96
cctatcattccc

SEQ ID NO: 100 - DNA, 12 bp, Artificial Sequence
  Synthetic- Halo_BC97
gaggtatgactc

SEQ ID NO: 101 - DNA, 12 bp, Artificial Sequence
  Synthetic- Halo_BC98
ctaggtcaagtc

SEQ ID NO: 102 - DNA, 12 bp, Artificial Sequence
  Synthetic- Halo_BC99
actcggctttca

SEQ ID NO: 103 - DNA, 12 bp, Artificial Sequence
  Synthetic- Halo_BC100
ttcacaagcgga

SEQ ID NO: 104 - DNA, 52 bp, Artificial Sequence
  Synthetic- linker
ccaccgctgagcaataactagtagtgacaggtcgtagatgagtcaacggcct

SEQ ID NO: 105 - DNA, 52 bp, Artificial Sequence
  Synthetic- linker
ccaccgctgagcaataactatctgtgaagtcccgtagatgagtcaacggcct

SEQ ID NO: 106 - DNA, 52 bp, Artificial Sequence
  Synthetic- linker
ccaccgctgagcaataactaatcagatcgcctcgtagatgagtcaacggcct

SEQ ID NO: 107 - DNA, 52 bp, Artificial Sequence
  Synthetic- linker
ccaccgctgagcaataactaaatgtggtctcgcgtagatgagtcaacggcct

SEQ ID NO: 108 - DNA, 52 bp, Artificial Sequence
  Synthetic- linker
ccaccgctgagcaataactacctctccaaacacgtagatgagtcaacggcct

SEQ ID NO: 109 - DNA, 52 bp, Artificial Sequence
  Synthetic- linker
ccaccgctgagcaataactatactggacaaggcgtagatgagtcaacggcct

SEQ ID NO: 110 - DNA, 52 bp, Artificial Sequence
  Synthetic- linker
ccaccgctgagcaataactatatcggagtcctcgtagatgagtcaacggcct

SEQ ID NO: 111 - DNA, 52 bp, Artificial Sequence
  Synthetic- linker
ccaccgctgagcaataactaggtggagttactcgtagatgagtcaacggcct

SEQ ID NO: 112 - DNA, 52 bp, Artificial Sequence
  Synthetic- linker
ccaccgctgagcaataactacggctactattgcgtagatgagtcaacggcct

SEQ ID NO: 113 - DNA, 52 bp, Artificial Sequence
  Synthetic- linker
ccaccgctgagcaataactaccgagctatgtacgtagatgagtcaacggcct

SEQ ID NO: 114 - DNA, 52 bp, Artificial Sequence
  Synthetic- linker
ccaccgctgagcaataactaactacgtccaaccgtagatgagtcaacggcct

SEQ ID NO: 115 - DNA, 52 bp, Artificial Sequence
  Synthetic- linker
ccaccgctgagcaataactattcatccgaacgcgtagatgagtcaacggcct

SEQ ID NO: 116 - DNA, 52 bp, Artificial Sequence
  Synthetic- linker
ccaccgctgagcaataactacgaaacgcttagcgtagatgagtcaacggcct

SEQ ID NO: 117 - DNA, 52 bp, Artificial Sequence
  Synthetic- linker
ccaccgctgagcaataactagcctaagttccacgtagatgagtcaacggcct

SEQ ID NO: 118 - DNA, 52 bp, Artificial Sequence; identical to SEQ ID NO: 117
  Synthetic- linker

SEQ ID NO: 119 - DNA, 52 bp, Artificial Sequence
  Synthetic- linker
ccaccgctgagcaataactacggtgagacatacgtagatgagtcaacggcct

SEQ ID NO: 120 - DNA, 52 bp, Artificial Sequence
  Synthetic- linker
ccaccgctgagcaataactactctgaggtttgcgtagatgagtcaacggcct

SEQ ID NO: 121 - DNA, 52 bp, Artificial Sequence
  Synthetic- linker
ccaccgctgagcaataactatactgtcacccacgtagatgagtcaacggcct

SEQ ID NO: 122 - DNA, 52 bp, Artificial Sequence
  Synthetic- linker
ccaccgctgagcaataactacaggaggtacatcgtagatgagtcaacggcct

SEQ ID NO: 123 - DNA, 52 bp, Artificial Sequence
  Synthetic- linker
ccaccgctgagcaataactacttcctacagcacgtagatgagtcaacggcct

SEQ ID NO: 124 - DNA, 52 bp, Artificial Sequence
  Synthetic- linker
ccaccgctgagcaataactatagaaaccgaggcgtagatgagtcaacggcct

SEQ ID NO: 125 - DNA, 52 bp, Artificial Sequence
  Synthetic- linker
ccaccgctgagcaataactagaaaagcgtacccgtagatgagtcaacggcct

SEQ ID NO: 126 - DNA, 52 bp, Artificial Sequence
  Synthetic- linker
ccaccgctgagcaataactacgctcataactccgtagatgagtcaacggcct

SEQ ID NO: 127 - DNA, 52 bp, Artificial Sequence
  Synthetic- linker
ccaccgctgagcaataactaggcatatacgaccgtagatgagtcaacggcct

SEQ ID NO: 128 - DNA, 52 bp, Artificial Sequence
  Synthetic- linker
ccaccgctgagcaataactagtgctctatcaccgtagatgagtcaacggcct

SEQ ID NO: 129 - DNA, 52 bp, Artificial Sequence
  Synthetic- linker
ccaccgctgagcaataactaggagcatttcaccgtagatgagtcaacggcct

SEQ ID NO: 130 - DNA, 52 bp, Artificial Sequence
  Synthetic- linker
ccaccgctgagcaataactaatgggtcttctgcgtagatgagtcaacggcct

SEQ ID NO: 131 - DNA, 52 bp, Artificial Sequence
  Synthetic- linker
ccaccgctgagcaataactaaagtccgtgaaccgtagatgagtcaacggcct

SEQ ID NO: 132 - DNA, 52 bp, Artificial Sequence
  Synthetic- linker
ccaccgctgagcaataactatgacatagagggcgtagatgagtcaacggcct

SEQ ID NO: 133 - DNA, 52 bp, Artificial Sequence
  Synthetic- linker
ccaccgctgagcaataactacgtcaatcgtgtcgtagatgagtcaacggcct

SEQ ID NO: 134 - DNA, 52 bp, Artificial Sequence
  Synthetic- linker
ccaccgctgagcaataactagttcgaagcaaccgtagatgagtcaacggcct

SEQ ID NO: 135 - DNA, 52 bp, Artificial Sequence
  Synthetic- linker
ccaccgctgagcaataactaacccgaattcaccgtagatgagtcaacggcct

SEQ ID NO: 136 - DNA, 52 bp, Artificial Sequence
  Synthetic- linker
ccaccgctgagcaataactagaggacttcacacgtagatgagtcaacggcct

SEQ ID NO: 137 - DNA, 52 bp, Artificial Sequence
  Synthetic- linker
ccaccgctgagcaataactagattccaccgtacgtagatgagtcaacggcct

SEQ ID NO: 138 - DNA, 52 bp, Artificial Sequence
  Synthetic- linker
ccaccgctgagcaataactagtattcgccatgcgtagatgagtcaacggcct

SEQ ID NO: 139 - DNA, 52 bp, Artificial Sequence
  Synthetic- linker
ccaccgctgagcaataactagcttgttatccgcgtagatgagtcaacggcct

SEQ ID NO: 140 - DNA, 52 bp, Artificial Sequence
  Synthetic- linker
ccaccgctgagcaataactacgtccaactatgcgtagatgagtcaacggcct

SEQ ID NO: 141 - DNA, 52 bp, Artificial Sequence
  Synthetic- linker
ccaccgctgagcaataactaggtaacagtgaccgtagatgagtcaacggcct

SEQ ID NO: 142 - DNA, 52 bp, Artificial Sequence
  Synthetic- linker
ccaccgctgagcaataactagcgcaaaagaagcgtagatgagtcaacggcct

SEQ ID NO: 143 - DNA, 52 bp, Artificial Sequence
  Synthetic- linker
ccaccgctgagcaataactatgtggttgatcgcgtagatgagtcaacggcct

SEQ ID NO: 144 - DNA, 52 bp, Artificial Sequence
  Synthetic- linker
ccaccgctgagcaataactatgtgggattgtgcgtagatgagtcaacggcct

SEQ ID NO: 145 - DNA, 52 bp, Artificial Sequence
  Synthetic- linker
ccaccgctgagcaataactatgcttcgggatacgtagatgagtcaacggcct

SEQ ID NO: 146 - DNA, 52 bp, Artificial Sequence
  Synthetic- linker
ccaccgctgagcaataactagacagctcgttacgtagatgagtcaacggcct

SEQ ID NO: 147 - DNA, 52 bp, Artificial Sequence
  Synthetic- linker
ccaccgctgagcaataactataagaagcgctccgtagatgagtcaacggcct

SEQ ID NO: 148 - DNA, 52 bp, Artificial Sequence
  Synthetic- linker
ccaccgctgagcaataactacatacacactcccgtagatgagtcaacggcct

SEQ ID NO: 149 - DNA, 52 bp, Artificial Sequence
  Synthetic- linker
ccaccgctgagcaataactatgccgccaaaatcgtagatgagtcaacggcct

SEQ ID NO: 150 - DNA, 52 bp, Artificial Sequence
  Synthetic- linker
ccaccgctgagcaataactacggaccttctaacgtagatgagtcaacggcct

SEQ ID NO: 151 - DNA, 52 bp, Artificial Sequence
  Synthetic- linker
ccaccgctgagcaataactatctcacgtcaaccgtagatgagtcaacggcct

SEQ ID NO: 152 - DNA, 52 bp, Artificial Sequence
  Synthetic- linker
ccaccgctgagcaataactacgcaagagaacacgtagatgagtcaacggcct

SEQ ID NO: 153 - DNA, 52 bp, Artificial Sequence
  Synthetic- linker
ccaccgctgagcaataactattagcttccctgcgtagatgagtcaacggcct

SEQ ID NO: 154 - DNA, 52 bp, Artificial Sequence
  Synthetic- linker
ccaccgctgagcaataactagaagccaagcatcgtagatgagtcaacggcct

SEQ ID NO: 155 - DNA, 52 bp, Artificial Sequence
  Synthetic- linker
ccaccgctgagcaataactattcgtagcgtgtcgtagatgagtcaacggcct

SEQ ID NO: 156 - DNA, 52 bp, Artificial Sequence
  Synthetic- linker
ccaccgctgagcaataactagtcgctgatcaacgtagatgagtcaacggcct

SEQ ID NO: 157 - DNA, 52 bp, Artificial Sequence
  Synthetic- linker
ccaccgctgagcaataactatcaactgatcggcgtagatgagtcaacggcct

SEQ ID NO: 158 - DNA, 52 bp, Artificial Sequence
  Synthetic- linker
ccaccgctgagcaataactaccagtttctacgcgtagatgagtcaacggcct

SEQ ID NO: 159 - DNA, 52 bp, Artificial Sequence
  Synthetic- linker
ccaccgctgagcaataactaacccattgcgatcgtagatgagtcaacggcct

SEQ ID NO: 160 - DNA, 52 bp, Artificial Sequence
  Synthetic- linker
ccaccgctgagcaataactatcaccaccctatcgtagatgagtcaacggcct

SEQ ID NO: 161 - DNA, 52 bp, Artificial Sequence
  Synthetic- linker
ccaccgctgagcaataactaggtcttcacttccgtagatgagtcaacggcct

SEQ ID NO: 162 - DNA, 52 bp, Artificial Sequence
  Synthetic- linker
ccaccgctgagcaataactagttagagatgggcgtagatgagtcaacggcct

SEQ ID NO: 163 - DNA, 52 bp, Artificial Sequence
  Synthetic- linker
ccaccgctgagcaataactatcttgcacactccgtagatgagtcaacggcct

SEQ ID NO: 164 - DNA, 52 bp, Artificial Sequence
  Synthetic- linker
ccaccgctgagcaataactattttctctgcggcgtagatgagtcaacggcct

SEQ ID NO: 165 - DNA, 52 bp, Artificial Sequence
  Synthetic- linker
ccaccgctgagcaataactatcagccgagttacgtagatgagtcaacggcct

SEQ ID NO: 166 - DNA, 52 bp, Artificial Sequence
  Synthetic- linker
ccaccgctgagcaataactactcgtgatcagacgtagatgagtcaacggcct

SEQ ID NO: 167 - DNA, 52 bp, Artificial Sequence
  Synthetic- linker
ccaccgctgagcaataactacctttctcggaacgtagatgagtcaacggcct

SEQ ID NO: 168 - DNA, 52 bp, Artificial Sequence
  Synthetic- linker
ccaccgctgagcaataactaacgctagagcttcgtagatgagtcaacggcct

SEQ ID NO: 169 - DNA, 52 bp, Artificial Sequence
  Synthetic- linker
ccaccgctgagcaataactattccccgtttagcgtagatgagtcaacggcct

SEQ ID NO: 170 - DNA, 52 bp, Artificial Sequence
  Synthetic- linker
ccaccgctgagcaataactaagaatcgcaacccgtagatgagtcaacggcct

SEQ ID NO: 171 - DNA, 52 bp, Artificial Sequence
  Synthetic- linker
ccaccgctgagcaataactaggaaggaactgtcgtagatgagtcaacggcct

SEQ ID NO: 172 - DNA, 52 bp, Artificial Sequence
  Synthetic- linker
ccaccgctgagcaataactacttggcatcttccgtagatgagtcaacggcct

SEQ ID NO: 173 - DNA, 52 bp, Artificial Sequence
  Synthetic- linker
ccaccgctgagcaataactaaggccgatttgtcgtagatgagtcaacggcct

SEQ ID NO: 174 - DNA, 52 bp, Artificial Sequence
  Synthetic- linker
ccaccgctgagcaataactaaacaaagggtcccgtagatgagtcaacggcct

SEQ ID NO: 175 - DNA, 52 bp, Artificial Sequence
  Synthetic- linker
ccaccgctgagcaataactacaattggtagcccgtagatgagtcaacggcct

SEQ ID NO: 176 - DNA, 52 bp, Artificial Sequence
  Synthetic- linker
ccaccgctgagcaataactaaccatcgactcacgtagatgagtcaacggcct

SEQ ID NO: 177 - DNA, 52 bp, Artificial Sequence
  Synthetic- linker
ccaccgctgagcaataactacgtgagatgaaccgtagatgagtcaacggcct

SEQ ID NO: 178 - DNA, 52 bp, Artificial Sequence
  Synthetic- linker
ccaccgctgagcaataactaccatggtcttgtcgtagatgagtcaacggcct

SEQ ID NO: 179 - DNA, 52 bp, Artificial Sequence
  Synthetic- linker
ccaccgctgagcaataactaagatatgagcgccgtagatgagtcaacggcct

SEQ ID NO: 180 - DNA, 52 bp, Artificial Sequence
  Synthetic- linker
ccaccgctgagcaataactagtgtgacagagtcgtagatgagtcaacggcct

SEQ ID NO: 181 - DNA, 52 bp, Artificial Sequence
  Synthetic- linker
ccaccgctgagcaataactaattgtgtgacggcgtagatgagtcaacggcct

SEQ ID NO: 182 - DNA, 52 bp, Artificial Sequence
  Synthetic- linker
ccaccgctgagcaataactacggtagtttgctcgtagatgagtcaacggcct

SEQ ID NO: 183 - DNA, 52 bp, Artificial Sequence
  Synthetic- linker
ccaccgctgagcaataactaggacatgtccatcgtagatgagtcaacggcct

SEQ ID NO: 184 - DNA, 52 bp, Artificial Sequence
  Synthetic- linker
ccaccgctgagcaataactattgagggagacacgtagatgagtcaacggcct

SEQ ID NO: 185 - DNA, 52 bp, Artificial Sequence
  Synthetic- linker
ccaccgctgagcaataactacgacatcctctacgtagatgagtcaacggcct

SEQ ID NO: 186 - DNA, 52 bp, Artificial Sequence
  Synthetic- linker
ccaccgctgagcaataactatgagcgagttcacgtagatgagtcaacggcct

SEQ ID NO: 187 - DNA, 52 bp, Artificial Sequence
  Synthetic- linker
ccaccgctgagcaataactagaccttcggattcgtagatgagtcaacggcct

SEQ ID NO: 188 - DNA, 52 bp, Artificial Sequence
  Synthetic- linker
ccaccgctgagcaataactatgtagatccgcacgtagatgagtcaacggcct

SEQ ID NO: 189 - DNA, 52 bp, Artificial Sequence
  Synthetic- linker
ccaccgctgagcaataactatggcactctagacgtagatgagtcaacggcct

SEQ ID NO: 190 - DNA, 52 bp, Artificial Sequence
  Synthetic- linker
ccaccgctgagcaataactaaacagtagtcggcgtagatgagtcaacggcct

SEQ ID NO: 191 - DNA, 52 bp, Artificial Sequence
  Synthetic- linker
ccaccgctgagcaataactatcatgcggaaagcgtagatgagtcaacggcct

SEQ ID NO: 192 - DNA, 52 bp, Artificial Sequence
  Synthetic- linker
ccaccgctgagcaataactatcgaatcgtgtccgtagatgagtcaacggcct

SEQ ID NO: 193 - DNA, 52 bp, Artificial Sequence
  Synthetic- linker
ccaccgctgagcaataactaggtgtatagccacgtagatgagtcaacggcct

SEQ ID NO: 194 - DNA, 52 bp, Artificial Sequence
  Synthetic- linker
ccaccgctgagcaataactattgcagtgcaagcgtagatgagtcaacggcct

SEQ ID NO: 195 - DNA, 52 bp, Artificial Sequence
  Synthetic- linker
ccaccgctgagcaataactacgattgcagaagcgtagatgagtcaacggcct

SEQ ID NO: 196 - DNA, 52 bp, Artificial Sequence
  Synthetic- linker
ccaccgctgagcaataactaccagacgttgttcgtagatgagtcaacggcct

SEQ ID NO: 197 - DNA, 52 bp, Artificial Sequence
  Synthetic- linker
ccaccgctgagcaataactatggtggccataacgtagatgagtcaacggcct

SEQ ID NO: 198 - DNA, 52 bp, Artificial Sequence
  Synthetic- linker
ccaccgctgagcaataactacagagtcaatggcgtagatgagtcaacggcct

SEQ ID NO: 199 - DNA, 52 bp, Artificial Sequence
  Synthetic- linker
ccaccgctgagcaataactacctatcattccccgtagatgagtcaacggcct

SEQ ID NO: 200 - DNA, 52 bp, Artificial Sequence
  Synthetic- linker
ccaccgctgagcaataactagaggtatgactccgtagatgagtcaacggcct

SEQ ID NO: 201 - DNA, 52 bp, Artificial Sequence
  Synthetic- linker
ccaccgctgagcaataactactaggtcaagtccgtagatgagtcaacggcct

SEQ ID NO: 202 - DNA, 52 bp, Artificial Sequence
  Synthetic- linker
ccaccgctgagcaataactaactcggctttcacgtagatgagtcaacggcct

SEQ ID NO: 203 - DNA, 52 bp, Artificial Sequence
  Synthetic- linker
ccaccgctgagcaataactattcacaagcggacgtagatgagtcaacggcct

SEQ ID NO: 204 - DNA, 70 bp, Artificial Sequence
  Synthetic- IndBCF1
aatgatacggcgaccaccgagatctacacgctatgattgcgtcctatggtaattgtaggccgttgactca

SEQ ID NO: 205 - DNA, 70 bp, Artificial Sequence
  Synthetic- IndBCF2
aatgatacggcgaccaccgagatctacacgcttgctcatcgatgtatggtaattgtaggccgttgactca

SEQ ID NO: 206 - DNA, 70 bp, Artificial Sequence
  Synthetic- IndBCF3
aatgatacggcgaccaccgagatctacacgctcacaggttctactatggtaattgtaggccgttgactca

SEQ ID NO: 207 - DNA, 70 bp, Artificial Sequence
  Synthetic- IndBCF4
aatgatacggcgaccaccgagatctacacgctctggcttgatcttatggtaattgtaggccgttgactca

SEQ ID NO: 208 - DNA, 70 bp, Artificial Sequence
  Synthetic- IndBCF5
aatgatacggcgaccaccgagatctacacgcttctctgtccgattatggtaattgtaggccgttgactca

SEQ ID NO: 209 - DNA, 70 bp, Artificial Sequence
  Synthetic- IndBCF6
aatgatacggcgaccaccgagatctacacgctcagccatggaaatatggtaattgtaggccgttgactca

SEQ ID NO: 210 - DNA, 70 bp, Artificial Sequence
  Synthetic- IndBCF7
aatgatacggcgaccaccgagatctacacgcttatgtaccggagtatggtaattgtaggccgttgactca

SEQ ID NO: 211 - DNA, 70 bp, Artificial Sequence
  Synthetic- IndBCF8
aatgatacggcgaccaccgagatctacacgctactgtaacgctctatggtaattgtaggccgttgactca

SEQ ID NO: 212 - DNA, 70 bp, Artificial Sequence
  Synthetic- IndBCF9
aatgatacggcgaccaccgagatctacacgctctagcgtccatttatggtaattgtaggccgttgactca

SEQ ID NO: 213 - DNA, 70 bp, Artificial Sequence
  Synthetic- IndBCF10
aatgatacggcgaccaccgagatctacacgcttggatatgccgatatggtaattgtaggccgttgactca

SEQ ID NO: 214 - DNA, 70 bp, Artificial Sequence
  Synthetic- IndBCF11
aatgatacggcgaccaccgagatctacacgctttccaacgttgctatggtaattgtaggccgttgactca

SEQ ID NO: 215 - DNA, 70 bp, Artificial Sequence
  Synthetic- IndBCF12
aatgatacggcgaccaccgagatctacacgctggtgtgaactcatatggtaattgtaggccgttgactca

SEQ ID NO: 216 - DNA, 70 bp, Artificial Sequence
  Synthetic- IndBCF13
aatgatacggcgaccaccgagatctacacgctcaaagggagatctatggtaattgtaggccgttgactca

SEQ ID NO: 217 - DNA, 70 bp, Artificial Sequence
  Synthetic- IndBCF14
aatgatacggcgaccaccgagatctacacgctctcacaatccgttatggtaattgtaggccgttgactca

SEQ ID NO: 218 - DNA, 70 bp, Artificial Sequence
  Synthetic- IndBCF15
aatgatacggcgaccaccgagatctacacgctggtgggtttgattatggtaattgtaggccgttgactca

SEQ ID NO: 219 - DNA, 70 bp, Artificial Sequence
  Synthetic- IndBCF16
aatgatacggcgaccaccgagatctacacgctccctttgtctagtatggtaattgtaggccgttgactca

SEQ ID NO: 220 - DNA, 70 bp, Artificial Sequence
  Synthetic- IndBCF17
aatgatacggcgaccaccgagatctacacgcttttctgctgagctatggtaattgtaggccgttgactca

SEQ ID NO: 221 - DNA, 70 bp, Artificial Sequence
  Synthetic- IndBCF18
aatgatacggcgaccaccgagatctacacgctacttctcctgcttatggtaattgtaggccgttgactca

SEQ ID NO: 222 - DNA, 70 bp, Artificial Sequence
  Synthetic- IndBCF19
aatgatacggcgaccaccgagatctacacgctccgaccataagatatggtaattgtaggccgttgactca

SEQ ID NO: 223 - DNA, 70 bp, Artificial Sequence
  Synthetic- IndBCF20
aatgatacggcgaccaccgagatctacacgctgactgctgatgatatggtaattgtaggccgttgactca

SEQ ID NO: 224 - DNA, 70 bp, Artificial Sequence
  Synthetic- IndBCF21
aatgatacggcgaccaccgagatctacacgctaatcgaggagagtatggtaattgtaggccgttgactca

SEQ ID NO: 225 - DNA, 70 bp, Artificial Sequence
  Synthetic- IndBCF22
aatgatacggcgaccaccgagatctacacgctagcgcactcttttatggtaattgtaggccgttgactca

SEQ ID NO: 226 - DNA, 70 bp, Artificial Sequence
  Synthetic- IndBCF23
aatgatacggcgaccaccgagatctacacgctaattgggtcgtctatggtaattgtaggccgttgactca

SEQ ID NO: 227 - DNA, 70 bp, Artificial Sequence
  Synthetic- IndBCF24
aatgatacggcgaccaccgagatctacacgcttcgttcggactatatggtaattgtaggccgttgactca

SEQ ID NO: 228 - DNA, 70 bp, Artificial Sequence
  Synthetic- IndBCF25
aatgatacggcgaccaccgagatctacacgctaacgtaatcgcgtatggtaattgtaggccgttgactca

SEQ ID NO: 229 - DNA, 70 bp, Artificial Sequence
  Synthetic- IndBCF26
aatgatacggcgaccaccgagatctacacgctcataggaacgcttatggtaattgtaggccgttgactca

SEQ ID NO: 230 - DNA, 70 bp, Artificial Sequence
  Synthetic- IndBCF27
aatgatacggcgaccaccgagatctacacgctgtcgacgcaaattatggtaattgtaggccgttgactca

SEQ ID NO: 231 - DNA, 70 bp, Artificial Sequence
  Synthetic- IndBCF28
aatgatacggcgaccaccgagatctacacgcttaaagtcctgggtatggtaattgtaggccgttgactca

SEQ ID NO: 232 - DNA, 70 bp, Artificial Sequence
  Synthetic- IndBCF29
aatgatacggcgaccaccgagatctacacgctgccgaacatacttatggtaattgtaggccgttgactca

SEQ ID NO: 233 - DNA, 70 bp, Artificial Sequence
  Synthetic- IndBCF30
aatgatacggcgaccaccgagatctacacgctcggattggtgtatatggtaattgtaggccgttgactca

SEQ ID NO: 234 - DNA, 63 bp, Artificial Sequence
  Synthetic- IndBCR1
caagcagaagacggcatacgagatctccttcatgacagtcagccagccccaccgctgagcaat

SEQ ID NO: 235 - DNA, 63 bp, Artificial Sequence
  Synthetic- IndBCR2
caagcagaagacggcatacgagatgaagatcgatggagtcagccagccccaccgctgagcaat

SEQ ID NO: 236 - DNA, 63 bp, Artificial Sequence
  Synthetic- IndBCR3
caagcagaagacggcatacgagataggaacagcgatagtcagccagccccaccgctgagcaat

SEQ ID NO: 237 - DNA, 63 bp, Artificial Sequence
  Synthetic- IndBCR4
caagcagaagacggcatacgagatccaatcgatacgagtcagccagccccaccgctgagcaat

SEQ ID NO: 238 - DNA, 63 bp, Artificial Sequence
  Synthetic- IndBCR5
caagcagaagacggcatacgagatatccaggagttcagtcagccagccccaccgctgagcaat

SEQ ID NO: 239 - DNA, 63 bp, Artificial Sequence
  Synthetic- IndBCR6
caagcagaagacggcatacgagataacaagccgaagagtcagccagccccaccgctgagcaat

SEQ ID NO: 240 - DNA, 63 bp, Artificial Sequence
  Synthetic- IndBCR7
caagcagaagacggcatacgagatagtgaggccataagtcagccagccccaccgctgagcaat

SEQ ID NO: 241 - DNA, 63 bp, Artificial Sequence
  Synthetic- IndBCR8
caagcagaagacggcatacgagattagacccactagagtcagccagccccaccgctgagcaat

SEQ ID NO: 242 - DNA, 63 bp, Artificial Sequence
  Synthetic- IndBCR9
caagcagaagacggcatacgagattagaggttgggtagtcagccagccccaccgctgagcaat

SEQ ID NO: 243 - DNA, 63 bp, Artificial Sequence
  Synthetic- IndBCR10
caagcagaagacggcatacgagattccccttctacaagtcagccagccccaccgctgagcaat

SEQ ID NO: 244 - DNA, 63 bp, Artificial Sequence
  Synthetic- IndBCR11
caagcagaagacggcatacgagataatccaacccctagtcagccagccccaccgctgagcaat

SEQ ID NO: 245 - DNA, 63 bp, Artificial Sequence
  Synthetic- IndBCR12
caagcagaagacggcatacgagatgctaagggttgaagtcagccagccccaccgctgagcaat

SEQ ID NO: 246 - DNA, 63 bp, Artificial Sequence
  Synthetic- IndBCR13
caagcagaagacggcatacgagatactgacgagtctagtcagccagccccaccgctgagcaat

SEQ ID NO: 247 - DNA, 63 bp, Artificial Sequence
  Synthetic- IndBCR14
caagcagaagacggcatacgagattgagttagtgcgagtcagccagccccaccgctgagcaat

SEQ ID NO: 248 - DNA, 63 bp, Artificial Sequence
  Synthetic- IndBCR15
caagcagaagacggcatacgagatggtatacacgtgagtcagccagccccaccgctgagcaat

SEQ ID NO: 249 - DNA, 63 bp, Artificial Sequence
  Synthetic- IndBCR16
caagcagaagacggcatacgagatctaggaggttcaagtcagccagccccaccgctgagcaat

SEQ ID NO: 250 - DNA, 63 bp, Artificial Sequence
  Synthetic- IndBCR17
caagcagaagacggcatacgagatcgttgttcctctagtcagccagccccaccgctgagcaat

SEQ ID NO: 251 - DNA, 63 bp, Artificial Sequence
  Synthetic- IndBCR18
caagcagaagacggcatacgagatcttgtcctcacaagtcagccagccccaccgctgagcaat

SEQ ID NO: 252 - DNA, 63 bp, Artificial Sequence
  Synthetic- IndBCR19
caagcagaagacggcatacgagatgtccaaagcaagagtcagccagccccaccgctgagcaat

SEQ ID NO: 253 - DNA, 63 bp, Artificial Sequence
  Synthetic- IndBCR20
caagcagaagacggcatacgagatgaacacatgagcagtcagccagccccaccgctgagcaat